SEQUENCE LISTING (157 sequences)

SEQ ID NO: 1 - protein, 774 aa, Artificial sequence
  Synthetic polypeptide
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYKRGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGT

SEQ ID NO: 2 - protein, 774 aa, Artificial sequence
  Synthetic polypeptide
MILDTDYITEDGKPVIRIFKKENGEFKIEYDRTFEPYLYALLKDDSAIEEVKKITAERHSTVVTVKRVEKVQKKFLGRSVEVWKLYFTHPQDVPAIMDKIREHPAVIDIYEYDIPFAIRYLIDKGLVPMEGDEELKLLALDIGTPCHEGEVFAEGPILMISYADEEGTRVITWRNVDLPYVDVLSTEREMIQRFLRVVKEKDPDVLITYNGDNFDFAYLKKRCEKLGINFTLGREGSEPKIQRMGDRFAVEVKGRIHFDLYPVIRRTVNLPIYTLEAVYEAVFGQPKEKVYAEEITTAWETGENLERVARYSMEDAKVTYELGKEFMPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYITMTIKEIEEKYGFKLIYSDTDGFFATIPGAEAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIENQVLPAVERILRAYGYRKEDLWYQKTRQVGLSARLKPKGT

SEQ ID NO: 3 - protein, 774 aa, Artificial sequence
  Synthetic polypeptide
MILDTDYITEDGKPVIRIFKKENGEFKIEYDRTFEPYLYALLKDDSAIEEVKKITAERHGTVVTVKRVEKVQKKFLGRPVEVWKLYFTHPQDVPAIMDKIREHPAVIDIYEYDIPFAIRYLIDKGLVPMEGDEELKLLAFDIETLYHEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRHQSHEGGYIKEPERGLWENIVYLDFRSLYPSIIITHNVSPDTLNREGCKEYDVAPQVGHRFCKDFPGFIPSLLGDLLEERQKIKKRMKATIDPIERKLLDYRQRAIKILANSLYGYYGYARARWYCKECAESVIAWGREYLTMTIKEIEEKYGFKVIYSDTDGFFATIPGADAETVKKKAMEFLKYINAKLPGALELEYEGFYKRGLFVTKKKYAVIDEEGKITTRGLEIVRRDWSEIAKETQARVLEALLKDGDVEKAVRIVKEVTEKLSKYEVPPEKLVIHKQITRDLKDYKATGPHVAVAKRLAARGVKIRPGTVISYIVLKGSGRIVDRAIPFDEFDPTKHKYDAEYYIEKQVLPAVERILRAFGYRKEDLRYQKTRQVGLSARLKPKGT

SEQ ID NO: 4 - DNA, 19 bp, Artificial sequence
  Synthetic primer
cgcagtagcggtaaacggc

SEQ ID NO: 5 - DNA, 22 bp, Artificial sequence
  Synthetic primer
gcggataacaatttcacacagg

SEQ ID NO: 6 - DNA, 53 bp, Artificial sequence
  Synthetic primer
cgcagtagcggtaaacggccgacggggaattctcacaggagacgagggggaaa

SEQ ID NO: 7 - DNA, 43 bp, Artificial sequence
  Synthetic primer
cgcagtagcggtaaacggcggagsagggygccagggggaagac

SEQ ID NO: 8 - DNA, 45 bp, Artificial sequence
  Synthetic primer
cgcagtagcggtaaacggcgctcagcgggaagaccttggggctgg

SEQ ID NO: 9 - DNA, 49 bp, Artificial sequence
  Synthetic primer
gcggataacaatttcacacaggttgragctcctcagaggagggygggaa

SEQ ID NO: 10 - DNA, 63 bp, Artificial sequence
  Synthetic primer
gcggataacaatttcacacaggctgctcatcagatggcgggaagatgaagacagatggtgcag

SEQ ID NO: 11 - DNA, 40 bp, Artificial sequence
  Synthetic primer
tattcccatggcgcgcccaggtccagctkgtrcagtctgg

SEQ ID NO: 12 - DNA, 40 bp, Artificial sequence
  Synthetic primer
tattcccatggcgcgcccaggtgcagctggtgsartctgg

SEQ ID NO: 13 - DNA, 39 bp, Artificial sequence
  Synthetic primer
tattcccatggcgcgcccagrtcaccttgaaggagtctg

SEQ ID NO: 14 - DNA, 38 bp, Artificial sequence
  Synthetic primer
tattcccatggcgcgccgaggtgcagctgktggagwcy

SEQ ID NO: 15 - DNA, 39 bp, Artificial sequence
  Synthetic primer
tattcccatggcgcgcccaggtgcagctgcaggagtcsg

SEQ ID NO: 16 - DNA, 39 bp, Artificial sequence
  Synthetic primer
tattcccatggcgcgcccaggtgcagctacagcagtggg

SEQ ID NO: 17 - DNA, 38 bp, Artificial sequence
  Synthetic primer
tattcccatggcgcgcccaggtacagctgcagcagtca

SEQ ID NO: 18 - DNA, 39 bp, Artificial sequence
  Synthetic primer
tattcccatggcgcgcctcaacacaacggttcccagtta

SEQ ID NO: 19 - DNA, 43 bp, Artificial sequence
  Synthetic primer
ggcgcgccatgggaatagccgacatccrgdtgacccagtctcc

SEQ ID NO: 20 - DNA, 43 bp, Artificial sequence
  Synthetic primer
ggcgcgccatgggaatagccgatattgtgmtgacbcagwctcc

SEQ ID NO: 21 - DNA, 43 bp, Artificial sequence
  Synthetic primer
ggcgcgccatgggaatagccgaaattgtrwtgacrcagtctcc

SEQ ID NO: 22 - DNA, 42 bp, Artificial sequence
  Synthetic primer
ggcgcgccatgggaatagccgaaacgacactcacgcagtctc

SEQ ID NO: 23 - DNA, 43 bp, Artificial sequence
  Synthetic primer
ggcgcgccatgggaatagcccagtctgtsbtgacgcagccgcc

SEQ ID NO: 24 - DNA, 40 bp, Artificial sequence
  Synthetic primer
ggcgcgccatgggaatagcccagcctgtgctgactcaryc

SEQ ID NO: 25 - DNA, 43 bp, Artificial sequence
  Synthetic primer
ggcgcgccatgggaatagcccagccwgkgctgactcagccmcc

SEQ ID NO: 26 - DNA, 41 bp, Artificial sequence
  Synthetic primer
ggcgcgccatgggaatagcccagtctgyyctgaytcagcct

SEQ ID NO: 27 - DNA, 42 bp, Artificial sequence
  Synthetic primer
ggcgcgccatgggaatagcctcctatgwgctgacwcagccaa

SEQ ID NO: 28 - DNA, 43 bp, Artificial sequence
  Synthetic primer
ggcgcgccatgggaatagcctcctctgagctgastcaggascc

SEQ ID NO: 29 - DNA, 43 bp, Artificial sequence
  Synthetic primer
ggcgcgccatgggaatagcctcctatgagctgayrcagcyacc

SEQ ID NO: 30 - DNA, 42 bp, Artificial sequence
  Synthetic primer
ggcgcgccatgggaatagccaattttatgctgactcagcccc

SEQ ID NO: 31 - DNA, 43 bp, Artificial sequence
  Synthetic primer
ggcgcgccatgggaatagcccagdctgtggtgacycaggagcc

SEQ ID NO: 32 - DNA, 35 bp, Artificial sequence
  Synthetic primer
  misc_feature (1)..(4): n is a, c, g, or t
nnnnatgggccctgsgatgggcccttggtggargc

SEQ ID NO: 33 - DNA, 34 bp, Artificial sequence
  Synthetic primer
  misc_feature (1)..(4): n is a, c, g, or t
nnnnatgggccctgggttggggcggatgcactcc

SEQ ID NO: 34 - DNA, 34 bp, Artificial sequence
  Synthetic primer
  misc_feature (1)..(4): n is a, c, g, or t
nnnnatgggccctgcttggggctggtcggggatg

SEQ ID NO: 35 - DNA, 34 bp, Artificial sequence
  Synthetic primer
  misc_feature (1)..(4): n is a, c, g, or t
nnnngtgcggccgcagatggtgcagccacagttc

SEQ ID NO: 36 - DNA, 34 bp, Artificial sequence
  Synthetic primer
  misc_feature (1)..(4): n is a, c, g, or t
nnnngtgcggccgcgagggygggaacagagtgac

SEQ ID NO: 37 - DNA, 69 bp, Artificial sequence
  Synthetic primer
  misc_feature (35)..(38): n is a, c, g, or t
gtctcgtgggctcggagatgtgtataagagacagnnnnatgggccctgsgatgggcccttggtggargc

SEQ ID NO: 38 - DNA, 68 bp, Artificial sequence
  Synthetic primer
  misc_feature (35)..(38): n is a, c, g, or t
gtctcgtgggctcggagatgtgtataagagacagnnnnatgggccctgggttggggcggatgcactcc

SEQ ID NO: 39 - DNA, 68 bp, Artificial sequence
  Synthetic primer
  misc_feature (35)..(38): n is a, c, g, or t
gtctcgtgggctcggagatgtgtataagagacagnnnnatgggccctgcttggggctggtcggggatg

SEQ ID NO: 40 - DNA, 67 bp, Artificial sequence
  Synthetic primer
  misc_feature (34)..(37): n is a, c, g, or t
tcgtcggcagcgtcagatgtgtataagagacagnnnngtgcggccgcagatggtgcagccacagttc

SEQ ID NO: 41 - DNA, 67 bp, Artificial sequence
  Synthetic primer
  misc_feature (34)..(37): n is a, c, g, or t
tcgtcggcagcgtcagatgtgtataagagacagnnnngtgcggccgcgagggygggaacagagtgac

SEQ ID NO: 42 - DNA, 49 bp, Artificial sequence
  Synthetic primer
aatgatacggcgaccaccgagatctacacgacgactcgtcggcagcgtc

SEQ ID NO: 43 - DNA, 45 bp, Artificial sequence
  Synthetic primer
caagcagaagacggcatacgagatgcctaagtctcgtgggctcgg

SEQ ID NO: 44 - DNA, 45 bp, Artificial sequence
  Synthetic primer
caagcagaagacggcatacgagattggtcagtctcgtgggctcgg

SEQ ID NO: 45 - DNA, 21 bp, Artificial sequence
  Synthetic primer
aggtgctcaacaacatggaga

SEQ ID NO: 46 - DNA, 25 bp, Artificial sequence
  Synthetic primer
ccccagtgctcacatggctgacttt

SEQ ID NO: 47 - DNA, 45 bp, Artificial sequence
  Synthetic primer
caagcagaagacggcatacgagatacatcggtctcgtgggctcgg

SEQ ID NO: 48 - DNA, 45 bp, Artificial sequence
  Synthetic primer
caagcagaagacggcatacgagattcaagtgtctcgtgggctcgg

SEQ ID NO: 49 - DNA, 45 bp, Artificial sequence
  Synthetic primer
caagcagaagacggcatacgagatctgatcgtctcgtgggctcgg

SEQ ID NO: 50 - DNA, 45 bp, Artificial sequence
  Synthetic primer
caagcagaagacggcatacgagattacaaggtctcgtgggctcgg

SEQ ID NO: 51 - DNA, 45 bp, Artificial sequence
  Synthetic primer
caagcagaagacggcatacgagattttcacgtctcgtgggctcgg

SEQ ID NO: 52 - DNA, 45 bp, Artificial sequence
  Synthetic primer
caagcagaagacggcatacgagataggaatgtctcgtgggctcgg

SEQ ID NO: 53 - DNA, 45 bp, Artificial sequence
  Synthetic primer
caagcagaagacggcatacgagatggaactgtctcgtgggctcgg

SEQ ID NO: 54 - DNA, 45 bp, Artificial sequence
  Synthetic primer
caagcagaagacggcatacgagatgctcatgtctcgtgggctcgg

SEQ ID NO: 55 - DNA, 45 bp, Artificial sequence
  Synthetic primer
caagcagaagacggcatacgagatccactcgtctcgtgggctcgg

SEQ ID NO: 56 - DNA, 45 bp, Artificial sequence
  Synthetic primer
caagcagaagacggcatacgagataacttcgtctcgtgggctcgg

SEQ ID NO: 57 - DNA, 45 bp, Artificial sequence; identical to SEQ ID NO: 56
  Synthetic primer

SEQ ID NO: 58 - DNA, 39 bp, Artificial sequence
  Synthetic primer
tattcccatggcgcgccggctcaaagccttctcagcagg

SEQ ID NO: 59 - DNA, 41 bp, Artificial sequence
  Synthetic primer
tattcccatggcgcgccggataacctggttaaaggcagcta

SEQ ID NO: 60 - DNA, 43 bp, Artificial sequence
  Synthetic primer
tattcccatggcgcgccggatacaagacaaaagttacaaacga

SEQ ID NO: 61 - DNA, 44 bp, Artificial sequence
  Synthetic primer
tattcccatggcgcgccgctgacgtatattttttcaaatatgga

SEQ ID NO: 62 - DNA, 40 bp, Artificial sequence
  Synthetic primer
tattcccatggcgcgccggaagaggccctgttttcttgct

SEQ ID NO: 63 - DNA, 41 bp, Artificial sequence
  Synthetic primer
tattcccatggcgcgccgctggatatgagaagcagaaagga

SEQ ID NO: 64 - DNA, 41 bp, Artificial sequence; identical to SEQ ID NO: 63
  Synthetic primer

SEQ ID NO: 65 - DNA, 42 bp, Artificial sequence
  Synthetic primer
tattcccatggcgcgccgtatgtccaatatcctggagaaggt

SEQ ID NO: 66 - DNA, 41 bp, Artificial sequence
  Synthetic primer
tattcccatggcgcgcccagtgagaacacaaagtcgaacgg

SEQ ID NO: 67 - DNA, 41 bp, Artificial sequence
  Synthetic primer
tattcccatggcgcgcccctaagttgctgatgtccgtatac

SEQ ID NO: 68 - DNA, 42 bp, Artificial sequence
  Synthetic primer
tattcccatggcgcgccgggaaaagccctgagttgataatgt

SEQ ID NO: 69 - DNA, 41 bp, Artificial sequence
  Synthetic primer
tattcccatggcgcgccgctgatgtacacatactccagtgg

SEQ ID NO: 70 - DNA, 41 bp, Artificial sequence
  Synthetic primer
tattcccatggcgcgcccccttggtataagcaagaacttgg

SEQ ID NO: 71 - DNA, 43 bp, Artificial sequence
  Synthetic primer
tattcccatggcgcgcccctcaattcattatagacattcgttc

SEQ ID NO: 72 - DNA, 41 bp, Artificial sequence
  Synthetic primer
tattcccatggcgcgccgcaaaatgcaacagaaggtcgcta

SEQ ID NO: 73 - DNA, 41 bp, Artificial sequence
  Synthetic primer
tattcccatggcgcgcctagagagagcatcaaaggcttcac

SEQ ID NO: 74 - DNA, 43 bp, Artificial sequence
  Synthetic primer
tattcccatggcgcgcccgttcaaatgaaagagagaaacacag

SEQ ID NO: 75 - DNA, 42 bp, Artificial sequence
  Synthetic primer
tattcccatggcgcgcccctgaaaagttcagaaaaccaggag

SEQ ID NO: 76 - DNA, 41 bp, Artificial sequence
  Synthetic primer
tattcccatggcgcgccggtcggtattcttggaacttccag

SEQ ID NO: 77 - DNA, 42 bp, Artificial sequence
  Synthetic primer
tattcccatggcgcgccgctggggaagaaaaggagaaagaaa

SEQ ID NO: 78 - DNA, 41 bp, Artificial sequence
  Synthetic primer
tattcccatggcgcgccgtcagagagagcaaacaagtggaa

SEQ ID NO: 79 - DNA, 41 bp, Artificial sequence; identical to SEQ ID NO: 78
  Synthetic primer

SEQ ID NO: 80 - DNA, 41 bp, Artificial sequence; identical to SEQ ID NO: 78
  Synthetic primer

SEQ ID NO: 81 - DNA, 43 bp, Artificial sequence
  Synthetic primer
tattcccatggcgcgccgactttaaatggggatgaaaagaaga

SEQ ID NO: 82 - DNA, 43 bp, Artificial sequence; identical to SEQ ID NO: 81
  Synthetic primer

SEQ ID NO: 83 - DNA, 41 bp, Artificial sequence
  Synthetic primer
tattcccatggcgcgccccaatgaaatggcctctctgatca

SEQ ID NO: 84 - DNA, 41 bp, Artificial sequence
  Synthetic primer
tattcccatggcgcgccgcaatgtgaacaacagaatggcct

SEQ ID NO: 85 - DNA, 41 bp, Artificial sequence
  Synthetic primer
tattcccatggcgcgccggtggagaagtgaagaagctgaag

SEQ ID NO: 86 - DNA, 44 bp, Artificial sequence
  Synthetic primer
tattcccatggcgcgccggataaaaatgaagatggaagattcac

SEQ ID NO: 87 - DNA, 42 bp, Artificial sequence
  Synthetic primer
tattcccatggcgcgcccctgatgatattactgaagggtgga

SEQ ID NO: 88 - DNA, 41 bp, Artificial sequence
  Synthetic primer
tattcccatggcgcgccggtggggaagagaaaagtcatgaa

SEQ ID NO: 89 - DNA, 42 bp, Artificial sequence
  Synthetic primer
tattcccatggcgcgccggtgaattgacctcaaatggaagac

SEQ ID NO: 90 - DNA, 43 bp, Artificial sequence
  Synthetic primer
tattcccatggcgcgccgctaacttcaagtggaattgaaaaga

SEQ ID NO: 91 - DNA, 43 bp, Artificial sequence
  Synthetic primer
tattcccatggcgcgccgaagcttataagcaacagaatgcaac

SEQ ID NO: 92 - DNA, 39 bp, Artificial sequence
  Synthetic primer
tattcccatggcgcgccggagcagtgaagcaggagggac

SEQ ID NO: 93 - DNA, 43 bp, Artificial sequence
  Synthetic primer
tattcccatggcgcgccgagagacaatggaaaacagcaaaaac

SEQ ID NO: 94 - DNA, 40 bp, Artificial sequence
  Synthetic primer
tattcccatggcgcgccgctgagctcagggaagaagaagc

SEQ ID NO: 95 - DNA, 44 bp, Artificial sequence
  Synthetic primer
ggcgcgccatgggaatactgaaatattcgatgatcaattctcag

SEQ ID NO: 96 - DNA, 45 bp, Artificial sequence
  Synthetic primer
ggcgcgccatgggaatatcattataaatgaaacagttccaaatcg

SEQ ID NO: 97 - DNA, 39 bp, Artificial sequence
  Synthetic primer
ggcgcgccatgggaataagtgtgccaagtcgcttctcac

SEQ ID NO: 98 - DNA, 41 bp, Artificial sequence
  Synthetic primer
ggcgcgccatgggaatacagaggaaactyccctcctagatt

SEQ ID NO: 99 - DNA, 44 bp, Artificial sequence
  Synthetic primer
ggcgcgccatgggaatagagacacagagaaacaaaggaaacttc

SEQ ID NO: 100 - DNA, 42 bp, Artificial sequence
  Synthetic primer
ggcgcgccatgggaataggtaccactgacaaaggagaagtcc

SEQ ID NO: 101 - DNA, 43 bp, Artificial sequence
  Synthetic primer
ggcgcgccatgggaatagagggtacaactgccaaaggagaggt

SEQ ID NO: 102 - DNA, 42 bp, Artificial sequence
  Synthetic primer
ggcgcgccatgggaataggcaaaggagaagtccctgatggtt

SEQ ID NO: 103 - DNA, 42 bp, Artificial sequence
  Synthetic primer
ggcgcgccatgggaataaaggagaagtcccsaatggctacaa

SEQ ID NO: 104 - DNA, 43 bp, Artificial sequence
  Synthetic primer
ggcgcgccatgggaatactgacaaagaagtccccaatggctac

SEQ ID NO: 105 - DNA, 42 bp, Artificial sequence
  Synthetic primer
ggcgcgccatgggaatacactgacaaaggagaagtccccgat

SEQ ID NO: 106 - DNA, 41 bp, Artificial sequence
  Synthetic primer
ggcgcgccatgggaataagacaaatcagggctgcccagtga

SEQ ID NO: 107 - DNA, 38 bp, Artificial sequence
  Synthetic primer
ggcgcgccatgggaatagactcagggctgcccaacgat

SEQ ID NO: 108 - DNA, 41 bp, Artificial sequence
  Synthetic primer
ggcgcgccatgggaataccagaatgaagctcaactagacaa

SEQ ID NO: 109 - DNA, 40 bp, Artificial sequence
  Synthetic primer
ggcgcgccatgggaataggttctctgcagagaggcctgag

SEQ ID NO: 110 - DNA, 39 bp, Artificial sequence
  Synthetic primer
ggcgcgccatgggaataggctgcccagtgatcggttctc

SEQ ID NO: 111 - DNA, 44 bp, Artificial sequence
  Synthetic primer
ggcgcgccatgggaatagacttacttccagaatgaagctcaact

SEQ ID NO: 112 - DNA, 45 bp, Artificial sequence
  Synthetic primer
ggcgcgccatgggaatagagcaaaaggaaacattcttgaacgatt

SEQ ID NO: 113 - DNA, 43 bp, Artificial sequence
  Synthetic primer
ggcgcgccatgggaataggctratccattactcatatggtgtt

SEQ ID NO: 114 - DNA, 42 bp, Artificial sequence
  Synthetic primer
ggcgcgccatgggaatagataaaggagaagtccccgatggct

SEQ ID NO: 115 - DNA, 42 bp, Artificial sequence
  Synthetic primer
ggcgcgccatgggaatagattcacagttgcctaaggatcgat

SEQ ID NO: 116 - DNA, 40 bp, Artificial sequence
  Synthetic primer
ggcgcgccatgggaatagattcagggatgcccgaggatcg

SEQ ID NO: 117 - DNA, 40 bp, Artificial sequence
  Synthetic primer
ggcgcgccatgggaatagattcggggatgccgaaggatcg

SEQ ID NO: 118 - DNA, 42 bp, Artificial sequence
  Synthetic primer
ggcgcgccatgggaatagcagagcgataaaggaagcatccct

SEQ ID NO: 119 - DNA, 42 bp, Artificial sequence
  Synthetic primer
ggcgcgccatgggaatatccggtatgcccaacaatcgattct

SEQ ID NO: 120 - DNA, 44 bp, Artificial sequence
  Synthetic primer
ggcgcgccatgggaatagattttaacaatgaagcagacacccct

SEQ ID NO: 121 - DNA, 43 bp, Artificial sequence
  Synthetic primer
ggcgcgccatgggaatagatgaaacaggtatgcccaaggaaag

SEQ ID NO: 122 - DNA, 45 bp, Artificial sequence
  Synthetic primer
ggcgcgccatgggaatatatcatagatgagtcaggaatgccaaag

SEQ ID NO: 123 - DNA, 44 bp, Artificial sequence
  Synthetic primer
ggcgcgccatgggaatagactttcagaaaggagatatagctgaa

SEQ ID NO: 124 - DNA, 42 bp, Artificial sequence
  Synthetic primer
ggcgcgccatgggaatacaaggccacatacgagcaaggcgtc

SEQ ID NO: 125 - DNA, 45 bp, Artificial sequence
  Synthetic primer
ggcgcgccatgggaatacaaagatataaacaaaggagagatctct

SEQ ID NO: 126 - DNA, 43 bp, Artificial sequence
  Synthetic primer
ggcgcgccatgggaataagagaagggagatctttcctctgagt

SEQ ID NO: 127 - DNA, 43 bp, Artificial sequence
  Synthetic primer
ggcgcgccatgggaatagactgataagggagatgttcctgaag

SEQ ID NO: 128 - DNA, 45 bp, Artificial sequence
  Synthetic primer
ggcgcgccatgggaataggctgatctatttctcatatgatgttaa

SEQ ID NO: 129 - DNA, 44 bp, Artificial sequence
  Synthetic primer
ggcgcgccatgggaatagccacatatgagagtggatttgtcatt

SEQ ID NO: 130 - DNA, 40 bp, Artificial sequence
  Synthetic primer
ggcgcgccatgggaataggtgccccagaatctctcagcct

SEQ ID NO: 131 - DNA, 27 bp, Artificial sequence
  Synthetic primer
accagtgtggccttttgggtgtgggag

SEQ ID NO: 132 - DNA, 27 bp, Artificial sequence
  Synthetic primer
accagtgtggccttttgggtgtgggag

SEQ ID NO: 133 - DNA, 59 bp, Artificial sequence
  Synthetic primer
  misc_feature (35)..(38): n is a, c, g, or t
gtctcgtgggctcggagatgtgtataagagacagnnnntacacggcagggtcagggttc

SEQ ID NO: 134 - DNA, 57 bp, Artificial sequence
  Synthetic primer
  misc_feature (34)..(37): n is a, c, g, or t
tcgtcggcagcgtcagatgtgtataagagacagnnnnatggctcaaacacagcgacc

SEQ ID NO: 135 - DNA, 23 bp, Artificial sequence
  Synthetic primer
caggtccagctkgtrcagtctgg

SEQ ID NO: 136 - DNA, 23 bp, Artificial sequence
  Synthetic primer
caggtgcagctggtgsartctgg

SEQ ID NO: 137 - DNA, 22 bp, Artificial sequence
  Synthetic primer
cagrtcaccttgaaggagtctg

SEQ ID NO: 138 - DNA, 21 bp, Artificial sequence
  Synthetic primer
gaggtgcagctgktggagwcy

SEQ ID NO: 139 - DNA, 22 bp, Artificial sequence
  Synthetic primer
caggtgcagctgcaggagtcsg

SEQ ID NO: 140 - DNA, 22 bp, Artificial sequence
  Synthetic primer
caggtgcagctacagcagtggg

SEQ ID NO: 141 - DNA, 21 bp, Artificial sequence
  Synthetic primer
caggtacagctgcagcagtca

SEQ ID NO: 142 - DNA, 22 bp, Artificial sequence
  Synthetic primer
tcaacacaacggttcccagtta

SEQ ID NO: 143 - DNA, 20 bp, Artificial sequence
  Synthetic primer
ggttggggcggatgcactcc

SEQ ID NO: 144 - DNA, 21 bp, Artificial sequence
  Synthetic primer
sgatgggcccttggtggargc

SEQ ID NO: 145 - DNA, 20 bp, Artificial sequence
  Synthetic primer
ggctcctgggggaagaagcc

SEQ ID NO: 146 - DNA, 54 bp, Artificial sequence
  Synthetic primer
  misc_feature (35)..(38): n is a, c, g, or t
gtctcgtgggctcggagatgtgtataagagacagnnnngcgccgcgatgggaat

SEQ ID NO: 147 - protein, 121 aa, Artificial sequence
  Synthetic polypeptide
EVQLVESGAEVKKPGESLRISCEGSGYSFTSYWISWVRQMPGKGLEWMGRIDPSDSYTNYGPSFQGHVTISVDKSISTAYLQWNSLKASDTAMYYCARPGGVTRDDAFDIWGQGTMVTVSS

SEQ ID NO: 148 - protein, 107 aa, Artificial sequence
  Synthetic polypeptide
DIRVTQSPSSLSASVGDRVTITCRASQSISGYLNWYQQKPGRPPKLLIYGASSLQSGVPSRFSGSGSGTDFTLTISSLQPEDFATYYCQQSYGTPGNFGQGTKLEIK

SEQ ID NO: 149 - protein, 123 aa, Artificial sequence
  Synthetic polypeptide
QVQLQESGPGLVKPSQTLSLTCTVSGDSITSGYYHWTWIRQHPGKGLEWIGYIYYSGSTDYNPSLKSRVIMSVDRSKNQFSLKLHSVTAADTAVYYCERGRPVAGTSPYFDSWGRGILVTVSS

SEQ ID NO: 150 - protein, 112 aa, Artificial sequence
  Synthetic polypeptide
QSVLTQPPSVSGAPGQRVTISCTGSSSNIGADYDVHWYQHLPGTAPKLLIYVSSNRPSGVPDRFSGSKSGTSASLAITGLQAEDEATYYCQSYDNTLSGSEVFGGGTKLTVL

SEQ ID NO: 151 - protein, 122 aa, Artificial sequence
  Synthetic polypeptide
QVQLVESGGGVVQPGTSLRLSCAVSGFTFSSYAMHWVRQAPGKGLEWVAVISHDGSSTYSPDSVKGRFTISRVISKNTVFLQMNSLRVEDTAVYYCAKDFLSAAISYGMDVWGQGTTVAVSS

SEQ ID NO: 152 - protein, 108 aa, Artificial sequence
  Synthetic polypeptide
SYELTQPPSVSVSPGQTARITCSGEALPNQYAYWYRQKPGQAPVLVIYKDTERPSGIPERFSGSSSRTAVTLTISGVQAEDEADYYCQSPHTSGTYVIFGGGTKLTVL

SEQ ID NO: 153 - protein, 121 aa, Artificial sequence
  Synthetic polypeptide
QVQLQESGPGLVRPSQTLSLTCTVSGDSVSSGGYSWNWIRQHPGKGLEWIGNIPYIGSANYNPSLKSRVSMSLDTSQNKFSLNLNFVTAADTAVYYCARDRGSYSRYFDLWGRGALVTVSS

SEQ ID NO: 154 - protein, 107 aa, Artificial sequence
  Synthetic polypeptide
DIRVTQSPTSVSASVGDRVTITCRASQYISRRLAWYQQRPGQAPKLLINAASSLQSGVPSRFSGSGSDRDFTLTIRSLEPEDSATYICQQADSFPLTFGGGTNVHVK

SEQ ID NO: 155 - protein, 122 aa, Artificial sequence
  Synthetic polypeptide
QVQLVESGGGLVKPGGSLRLSCAASGFNFNDYYMTWIRQAPGKGLEWLAYISGRTSFTKYADSVKGRFTISKDNAKKTLSLQMNTVRAEDTAVYYCGRLGDFWSGSESLDIWGQGTVVTVSP

SEQ ID NO: 156 - protein, 110 aa, Artificial sequence
  Synthetic polypeptide
QPVLTQPPSASGTPGQRVVISCTGAKSNIGTNTVNWYQQFPGTAPKLLIYNNDQRPSGVPDRFSGSRSGTSGSLAISGLQSEDEADYHCATWDDSVNGPVFGGGTKLTVL

SEQ ID NO: 157 - protein, 32 aa, Artificial sequence
  Synthetic polypeptide
CARGLLRGGWNDVDYYGNDVWCQQSYSTPLTF

(19) United States
(12) Patent Application Publication
(10) Pub. No.: US 2020/0216840 A1
(43) Pub. Date: Jul. 9, 2020

(54) AMPLIFICATION OF PAIRED PROTEIN-CODING MRNA SEQUENCES

(71) Applicant: BOARD OF REGENTS, THE UNIVERSITY OF TEXAS SYSTEM, Austin, TX (US)

(72) Inventors: Hidetaka TANNO, Austin, TX (US); George GEORGIOU, Austin, TX (US); Jonathan MCDANIEL, Austin, TX (US); Gregory IPPOLITO, Austin, TX (US); Andrew ELLINGTON, Austin, TX (US)

(21) Appl. No.: 16/633,981

(22) PCT Filed: Jul. 27, 2018
(86) PCT No.: PCT/US18/44171

§ 371 Date: Jan. 24, 2020

Related U.S. Application Data
(60) Provisional application No. 62/537,686, filed on Jul. 27, 2017.

(51) Int. Cl. C12N 15/10; G01N 1/28; C12Q 1/6874; C12N 9/12

(52) U.S. Cl. CPC C12N 15/1096; G01N 1/28; C12Y 207/07007; C12Q 1/6874; C12N 9/1252; C12N 15/1006

(57) ABSTRACT

The present disclosure generally relates to sequencing two or more genes expressed in a single cell in a high-throughput manner using reverse transcriptases. More particularly, the present disclosure relates to a method for high-throughput sequencing of pairs of transcripts co-expressed in single cells (e.g., antibody VH and VL coding sequence) to determine pairs of polypeptide chains that comprise immune receptors.

This application claims the benefit of U.S. Provisional Patent Application No. 62/537,686, filed Jul. 27, 2017, the entirety of which is incorporated herein by reference.

This invention was made with government support under Grant No. HDTRA1-12-C-0105 awarded by the Department of Defense/Department of Threat Reduction. The government has certain rights in the invention.

### BACKGROUND OF THE INVENTION

### 1. Field of the Invention

The present invention relates generally to the field of molecular biology. More particularly, it concerns amplification of paired protein-coding mRNA sequences using a modified DNA polymerase having reverse transcriptase activity.

### 2. Description of Related Art

There is a need to identify the expression of two or more transcripts from individual cells at high throughput. In particular, for numerous biotechnology and medical applications it is important to identify and sequence the gene pairs encoding the two chains comprising adaptive immune receptors from individual cells at a very high throughput in order to accurately determine the complete repertoires of immune receptors expressed in patients or in laboratory animals. Immune receptors expressed by B and T lymphocytes are encoded respectively by the VH and VL antibody genes and by TCR α/β or γ/δ chain genes. Humans have many tens of thousands or millions of distinct B and T lymphocytes classified into different subsets based on the expression of surface markers (CD proteins) and transcription factors (e.g., FoxP3 in the Treg T lymphocyte subset). High-throughput DNA sequencing technologies have been used to determine the repertoires of VH or VL chains or, alternatively, of TCR α and β in lymphocyte subsets of relevance to particular disease states or, more generally, to study the function of the adaptive immune system (Wu et al., 2011). Immunology researchers have an especially great need for high throughput analysis of multiple transcripts at once.

Currently available methods for immune repertoire sequencing involve mRNA isolation from a cell population of interest, e.g., memory B-cells or plasma cells from bone marrow, followed by RT-PCR in bulk to synthesize cDNA for high-throughput DNA sequencing (Reddy et al., 2010; Krause et al., 2011). However, heavy and light antibody chains (or a and β T-cell receptors) are encoded on separate mRNA strands and must be sequenced separately. Thus, these available methods have potential to unveil the entire heavy and light chain immune repertoires individually, but cannot yet resolve heavy and light chain pairings at high throughput. Without multiple-transcript analysis at the single-cell level to collect heavy and light chain pairing data, the full adaptive immune receptor, which includes both chains, cannot be sequenced or reconstructed and expressed for further study.

### SUMMARY OF THE INVENTION

In one embodiment, compositions isolated in a compartment are provided, said compositions comprising (i) polymerase that comprises one or more genetically engineered mutations compared to a wild-type Archaeal Family-B polymerase, the polymerase having an amino acid sequence at least 70%, 75%, 80%, 85%, 90%, 95%, 97%, 98%, or 99% identical to SEQ ID NO: 1 and in which one or more amino acid residues at a position selected from the group consisting of positions Y493, Y384, V389, 1521, E664 and G711 in the amino acid sequence shown in SEQ ID NO: 1 or at a position corresponding to any of these positions, are substituted with another amino acid residue; and (ii) a DNA molecule comprising linked cDNAs corresponding to two distinct mRNA transcripts from a single cell. In some aspects, the compartment is an emulsion macrovesicle. In certain aspects, the two distinct mRNA transcripts encode paired antibody VH and VL domains. In other aspects, the two distinct mRNA transcripts encode paired T-cell receptor sequences.

In one embodiment, methods are provided, said methods comprising: a) sequestering single cells into individual compartments; b) lysing the cells to generate a lysate comprising mRNA transcripts; c) performing reverse transcription and a first PCR amplification of the mRNA transcripts using a single polymerase to generate distinct cDNA products corresponding to at least two distinct mRNAs from a single cell; and d) sequencing the distinct cDNA products amplified from at least one single cell. In some aspects, the single polymerase has proofreading activity. In certain aspects, the methods is further defined as a method for obtaining a plurality of natively paired mRNA transcript sequences.

In some aspects, the cells are B cells. In certain aspects, the at least two distinct mRNAs encode paired antibody VH and VL sequences. As such, the method may be further defined as a method for obtaining paired antibody VH and VL sequences for an antibody that binds to an antigen of interest.

In some aspects, the cells are T cells. In certain aspects, the at least two distinct mRNAs encode paired T-cell receptor sequences. As such, the method may be further defined as a method for obtaining paired T-cell receptor sequences for a T-cell receptor that binds to an epitope of interest.

In certain aspects, the mRNA transcripts are not captured. In certain aspects, the mRNA transcripts are bound to a solid support prior to step (c). As such, the method may further comprise binding the mRNA transcripts to a solid support prior to step (c). In some aspects, the solid support is a bead. In certain aspects, the solid support comprises oligonucleotides that hybridize to the mRNA transcripts, such as, for example, oligonucleotides comprising poly-T sequences.

In some aspects, the individual compartments are wells in a gel or microtiter plate. In certain aspects, the individual compartments have a volume of greater than 5 nL. In further aspects, the wells are sealed with a permeable membrane prior to step (c). In some aspects, the individual compartments are microvesicles in an emulsion.

In some aspects, steps (a) and (b) are performed concurrently. In certain aspects, steps (a) and (b) comprise isolating single cells into individual microvesicles in an emulsion and in the presence of a cell lysis solution.

In some aspects, the individual compartments in step (a) further comprise oligonucleotides for priming of reverse transcription. In certain aspects, step (b) further comprises allowing the mRNA transcripts to associate with the oligonucleotides. In certain aspects, the method comprises obtaining sequences from at least 10,000 individual cells. In certain aspects, the method comprises obtaining at least 5,000 individual paired antibody VH and VL sequences.

In some aspects, step (c) comprises linking cDNA by performing overlap extension reverse transcriptase polymerase chain reaction to link at least two transcripts into a single DNA molecule. In some aspects, step (c) does not comprise the use of overlap extension reverse transcriptase polymerase chain reaction. In some aspects, step (c) comprises linking VH and VL cDNAs by performing overlap extension reverse transcriptase polymerase chain reaction to link VH and VL cDNAs in single molecules. In certain aspects, step (c) does not comprise the use of overlap extension reverse transcriptase polymerase chain reaction and wherein the VH and VL cDNAs are separate molecules. In certain aspects, the VH and VL sequences are obtained by sequencing of distinct molecules. As such, the method may further comprise identifying the paired antibody VH and VL sequences comprises performing a probability analysis of the sequences. In some aspects, the probability analysis is based on the CDR-H3 or CDR-L3 sequences. In some aspects, identifying the paired antibody VH and VL sequences comprises comparing raw sequencing read counts.

In some aspects, step (c) comprises linking cDNA by performing recombination. In some aspects, the methods further comprise performing a second PCR amplification after step (c) and before step (d).

In some aspects, the cells are mammalian cells. In certain aspects, the cells are B cells, T cells, NKT cells, or cancer cells.

In some aspects, sequestering the single cells comprises introducing the cells to a device comprising a plurality of microwells so that the majority of cells are captured as single cells. In some aspects, the methods further comprise identifying multiple mRNA transcripts for a plurality of single cells based on the sequencing step (d). In some aspects, the methods further comprise isolating the mRNA transcripts prior to step (c). In some aspects, the methods further comprise determining natively paired transcripts using probability analysis. In certain aspects, identifying the natively paired transcripts comprises comparing raw sequencing read counts.

In various aspects of the present embodiments, the single polymerase is a recombinant Archaeal Family-B polymerase that transcribes a template that is RNA and has one or more mutations compared to a wild-type Archaeal Family-B polymerase. The polymerase may have one or more mutations compared to wild-type KOD polymerase. The one or more mutations are in a region of the polymerase that induces stalling at uracil residues; one or more mutations are in a region that recognizes the 2′ hydroxyl of template RNAs; one or more mutations are in a region that directly acts with a template strand; one or more mutations are in a region for secondary shell interactions; one or more mutations are in a template recognition interface region; one or more mutations are in a region for recognizing an incoming template; one or more mutations are in an active site region; and/or one or more mutations are in a post-polymerization region, in specific embodiments. In some cases, a mutation is in a region or position in which the polymerase recognizes the 2′ hydroxyl of a template RNA. At least one mutation may be an amino acid substitution, in at least some cases.

In some aspects, the polymerase has one or more genetically engineered mutations compared to a wild-type Archaeal Family-B polymerase, the polymerase having an amino acid sequence at least 70%, 75%, 80%, 85%, 90%, 95%, 97%, 98%, or 99% identical to SEQ ID NO: 1 and in which one or more amino acid residues at a position selected from the group consisting of positions Y493, Y384, V389, 1521, E664 and G711 in the amino acid sequence shown in SEQ ID NO:1 or at a position corresponding to any of these positions, are substituted with another amino acid residue. In some cases, the polymerase comprises an amino acid substitution corresponding to position Y493 to a leucine residue or a cysteine residue. In some cases, the polymerase comprises an amino acid substitution corresponding to position Y493 to a leucine residue. In some cases, the polymerase comprises an amino acid substitution corresponding to position Y384 to a phenylalanine residue, a leucine residue, an alanine residue, a cysteine residue, a serine residue, a histidine residue, an isoleucine residue, a methionine residue, an asparagine residue, or a glutamine residue. In some cases, the polymerase comprises an amino acid substitution corresponding to position Y384 to a histidine residue or an isoleucine residue. In some cases, the polymerase comprises an amino acid substitution corresponding to position V389 to a methionine residue, a phenylalanine residue, a threonine residue, a tyrosine residue, a glutamine residue, an asparagine residue, or a histidine residue. In some cases, the polymerase comprises an amino acid substitution corresponding to position V389 to an isoleucine residue. In some cases, the polymerase comprises an amino acid substitution corresponding to position 1521 to a leucine. In some cases, the polymerase comprises an amino acid substitution corresponding to E664 is to a lysine residue. In some cases, the polymerase comprises an amino acid substitution corresponding to position G711 to a leucine residue, a cysteine residue, a threonine residue, an arginine residue, a histidine residue, a glutamine residue, a lysine residue, or a methionine residue. In some cases, the polymerase comprises an amino acid substitution corresponding to position G711 to a valine residue. In some cases, the polymerase comprises an amino acid substitution at a position R97 in the amino acid sequence shown in SEQ ID NO:1 with another amino acid residue. In some cases, the polymerase comprises one or more amino acid residues at a position selected from the group consisting of positions A490, F587, M137, K118, T514, R381, F38, K466, E734 and N735 in the amino acid sequence shown in SEQ ID NO:1 or at a position corresponding to any of these positions, which is substituted with another amino acid residue. In some cases, the polymerase has proofreading activity. In some cases, the polymerase lacks proofreading activity. In some cases, the polymerase has thermophilic activity. In some cases, the polymerase is capable transcribing at least 10 nucleotides from a RNA template. In some cases, the polymerase is capable of transcribing a template that is 2′-OMethyl DNA. In some cases, the polymerase is capable transcribing at least 5 or at least 10 nucleotides from a 2′-OMethyl DNA template.

In some aspects, the polymerase has an amino acid sequence that is at least 70%, 75%, 80%, 85%, 90%, 95%, 97%, 98%, or 99% identical to the amino acid sequence of SEQ ID NO:1 and an amino acid substitution corresponding to an amino acid at positions 493, 384, 389, 97, 521, 711, 735, or a combination thereof. In some cases, the polymerase further comprises an amino acid substitution corresponding to an amino acid at positions 664. In some cases, the polymerase further comprises an amino acid substitution corresponding to position 493 to a leucine residue, a cysteine residue, or a phenylalanine residue. In some cases, the polymerase further comprises an amino acid substitution corresponding to position 493 to a leucine residue. In some cases, the polymerase further comprises an amino acid substitution corresponding to position 493 to an isoleucine residue, a valine residue, an alanine residue, a histidine residue, a threonine residue, or a serine residue. In some cases, the polymerase has an amino acid sequence that is at least 70%, 75%, 80%, 85%, 90%, 95%, 97%, 98%, or 99% identical to the amino acid sequence of SEQ ID NO:1 and an amino acid substitution corresponding to an amino acid at positions 493, 384, 389, 521, 711 or a combination thereof. In some cases, the polymerase comprises an amino acid substitution that corresponds to an amino acid at position 490, 587, 137, 118, 514, 381, 38, 466, 734, ora combination thereof. In some cases, the polymerase comprises an amino acid substitution corresponding to position 384 to a histidine residue or an isoleucine residue. In some cases, the polymerase comprises an amino acid substitution corresponding to position 384 to a phenylalanine residue, a leucine residue, an alanine residue, a cysteine residue, a serine residue, a histidine residue, an isoleucine residue, a methionine residue, an asparagine residue, or a glutamine residue. In some cases, the polymerase comprises an amino acid substitution corresponding to position 389 to an isoleucine residue or a leucine residue. In some cases, the polymerase comprises an amino acid substitution corresponding to position 389 to a methionine residue, a phenylalanine residue, a threonine residue, a tyrosine residue, a glutamine residue, an asparagine residue, or a histidine residue. In some cases, the amino acid substitution corresponding to position 664 is to a lysine residue or a glutamine residue. In some cases, the amino acid substitution corresponding to position 97 to any amino acid residue other than arginine. In some cases, the amino acid substitution corresponding to position 521 to a leucine. In some cases, the amino acid substitution corresponding to position 521 to a phenylalanine residue, a valine residue, a methionine residue, or a threonine residue. In some cases, the amino acid substitution corresponding to position 711 to a valine residue, a serine residue, or an arginine residue. In some cases, the amino acid substitution corresponding to position 711 to a leucine residue, a cysteine residue, a threonine residue, an arginine residue, a histidine residue, a glutamine residue, a lysine residue, or a methionine residue. In some cases, the amino acid substitution corresponding to position 735 to a lysine residue. In some cases, the amino acid substitution corresponding to position 735 to an arginine residue, a glutamine residue, an arginine residue, a tyrosine residue, or a histidine residue. In some cases, the amino acid substitution corresponding to position 490 is to a threonine residue. In some cases, the amino acid substitution corresponding to position 490 is to a valine residue, a serine residue, or a cysteine residue. In some cases, the amino acid substitution corresponding to position 587 is to a leucine residue or an isoleucine residue. In some cases, the amino acid substitution corresponding to position 587 is to an alanine residue, a threonine residue, or a valine residue. In some cases, the amino acid substitution corresponding to position 137 is to a leucine residue or an isoleucine residue. In some cases, the amino acid substitution corresponding to position 137 is to an alanine residue, a threonine residue, or a valine residue. In some cases, the amino acid substitution corresponding to position 118 is to an isoleucine residue. In some cases, the amino acid substitution corresponding to position 118 is to a methionine residue, a valine residue, or a leucine residue. In some cases, the amino acid substitution corresponding to position 514 is to an isoleucine residue. In some cases, the amino acid substitution corresponding to position 514 is to a valine residue, a leucine residue, or a methionine residue. In some cases, the amino acid substitution corresponding to position 381 is to a histidine residue. In some cases, the amino acid substitution corresponding to position 381 is to a serine residue, a glutamine residue, or a lysine residue. In some cases, the amino acid substitution corresponding to position 38 is to a leucine residue or an isoleucine residue. In some cases, the amino acid substitution corresponding to position 38 is to a valine residue, a methionine residue, or a serine residue. In some cases, the amino acid substitution corresponding to position 466 is to an arginine residue. In some cases, the amino acid substitution corresponding to position 466 is to a glutamate residue, an aspartate residue, or a glutamine residue. In some cases, the amino acid substitution corresponding to position 734 is to a lysine residue. In some cases, the amino acid substitution corresponding to position 734 is to an arginine residue, a glutamine residue, or an asparagine residue.

In certain aspects, the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution at one or more of the following positions corresponding to SEQ ID NO:1: R97; Y384; V389; Y493; F587; E664; G711; and W768. In some cases, the polymerase has one or more of the following amino acid substitutions corresponding to SEQ ID NO:1: R97M; Y384H; V389I; Y493L; F587L; E664K; G711V; and W768R.

In certain aspects, the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution at one or more of the following positions corresponding to SEQ ID NO:1: F38; R97; K118; R381; Y384; V389; Y493; T514; F587; E664; G711; and W768. In some cases, the polymerase has one or more of the following amino acid substitutions corresponding to SEQ ID NO:1: F38L; R97M; K1181; R381H; Y384H; V389I; Y493L; T514I; F587L; E664K; G711V; and W768R.

In certain aspects, the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution at one or more of the following positions corresponding to SEQ ID NO:1: F38; R97; K118; M137; R381; Y384; V389; K466; Y493; T514; F587; E664; G711; and W768. In some cases, the polymerase has one or more of the following amino acid substitutions corresponding to SEQ ID NO:1: F38L; R97M; K1181; M137L; R381H; Y384H; V389I; K466R; Y493L; T514I; F587L; E664K; G711V; and W768R.

In certain aspects, the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution at one or more of the following positions corresponding to SEQ ID NO:1: F38; R97; K118; M137; R381; Y384; V389; K466; Y493; T514; 1521; F587; E664; G711; N735; and W768. In some cases, the polymerase has one or more of the following amino acid substitutions corresponding to SEQ ID NO:1: F38L; R97M; K1181; M137L; R381H; Y384H; V389I; K466R; Y493L; T514I; I521L; F587L; E664K; G711V; N735K; and W768R.

In certain cases, polymerases further comprise an additional domain, such as one that does not itself take part in polymerization but has polymerization enhancing activity. In a specific embodiment, the additional domain comprise part or all of DNA-binding protein 7d (Sso7d), Proliferating cell nuclear antigen (PCNA), helicase, single stranded binding proteins, bovine serum albumin (BSA), one or more affinity tags, a label, and a combination thereof.

In certain aspects, the polymerase lacks 3′ to 5′ exonuclease activity. In some cases, the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution corresponding to N210. In some cases, the polymerase has an amino acid substitution corresponding to N210D. In some cases, the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution corresponding to D141 and E143. In some cases, the polymerase has an amino acid substitution corresponding to D141A and E143A.

In certain aspects, the polymerase comprises an amino acid sequence 98% identical to the amino acid sequence of SEQ ID NO: 3. In certain aspects, the polymerase comprises an amino acid sequence 99% identical to the amino acid sequence of SED ID NO: 3. In one aspect, the polymerase comprises an amino acid sequence identical to the amino acid sequence of SEQ ID NO: 3.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Flow-joint apparatus schematic. One syringe contains viable cells, and the other syringe contains 2×RT-PCR reagent consisting of RTX polymerase, overlap-extension primers, dNTPs, Betaine, polymerase buffer, BSA, Superaseln, and detergent. The two syringes are simultaneously compressed by the syringe pump to merge the cells and the RT-PCR solution at the junction. The rapidly flowing aqueous phase is emulsified by forcing the stream through a needle into a well-mixed oil phase. Single water-in-oil emulsions contain lysate from cells and RT-PCR solution.

FIG. 2. Overlap extension (OE) RT-PCR. i) Antibody heavy chain and light chain mRNA transcripts (comprising V, (D), J, and C regions) are reverse transcribed from constant region (CR) primers. ii) In the initial phase of the PCR reaction, individual VH and VL (or TCRa and TCRI3) genes are amplified using a multiplex set of OE V-region primers and constant region primers. iii) Once the individual VH and VL transcripts reach a critical concentration within each emulsion, the complementary linking regions are joined to generate a VH:VL amplicon. iv) The final amplicon represents the fusion of the VH and VL cDNAs. Newly synthesized DNAs are indicated by broken lines.

FIG. 3. RTX efficiently generates VH:VL fusion amplicons in the presence of cell lysate in the emulsion while other RT-PCR kits do not. One million total B cells were lysed with RT-PCR reagents containing surfactant and then emulsified. The resulting emulsions were subjected to overlap extension RT-PCR. The 850 bp VH:VL fusion cDNAs were detected by following Nested PCR. NC: Negative control. Emu: Emulsion RT-PCR with cell lysate. PC: Positive control using total B cell RNA.

FIGS. 4A-E. Technical replicates of VH:VL pairing experiment. FIG. 4A) Rarefaction analysis was used to calculate the number of B cell lineages in each experiment. The technical replicates demonstrate a high level of consistency with regards to CDRH3 length (FIG. 4B) and V-gene usage (FIG. 4C) (Spearman correlation p=0.99). FIG. 4D) Number of lineages identified and the mean CDRH3 length from each experiment. FIG. 4E) After spiking a healthy human sample of peripheral B cells with an ARH-77 cell line, this procedure was able to correctly identify the CDRH3:CDRL3 pair from each data set. (SEQ ID NO: 157)

FIGS. 5A-B. RTX efficiently generates PGK1 cDNA in the presence of cell lysate while other RT-PCR kits do not. FIG. 5A) Various RT-PCR kits supplemented with detergent were mixed with 2×10⁴ HEK293 cells. RT-PCR for PGK1 mRNA was conducted. As a positive control, 300 ng HEK293 total RNA was used. NTC: no template control; SS3: SuperScriptIII kit. FIG. 5B) Various RT-PCR kits supplemented with detergent were mixed with 2×10⁴ HEK293 cells and RT-PCR for PGK1 mRNA was conducted. Initial 65° C. heating step was added to lysis the cells. NTC: no template control; SS3: SuperScriptIII kit. Of note, the Titan system is a kit designed for cell lysate resistance RT-PCR, see e.g., Rajan et al. 2018, incorporated herein by reference.

FIGS. 6A-B. Photograph of entire setup. FIG. 6A) One syringe contains viable cells and another syringe contains RT-PCR reagent supplemented with detergent. The syringes are compressed by the syringe pump and resulting stream is immediately emulsified by the disperser. FIG. 6B) A structure of flow-joint apparatus. Two aqueous flows merge at the Y junction.

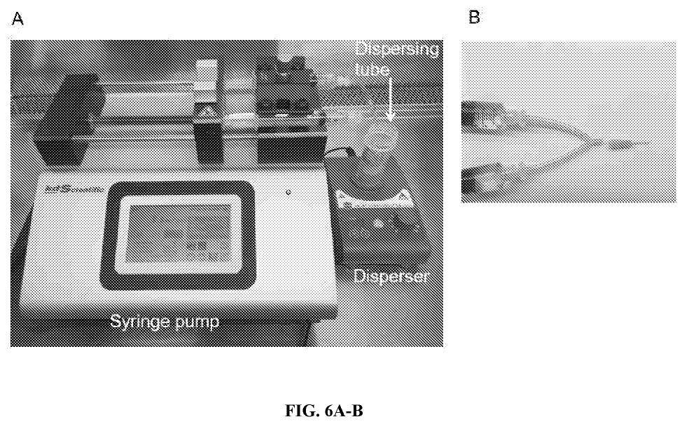

FIG. 7. FACS sorting of plasmablasts and memory B cells from the Fluzone vaccinated donor. The PBMCs freshly drawn from the Fluzone® vaccinee were stained with anti-human CD19-v450 (HIB19, BD Biosciences, San Jose, Calif.), CD27-APC (M-T271, BD Biosciences), CD38-PE (HIT2, BioLegend, San Diego, Calif.), CD20-FITC (2H7, BioLegend), and CD3-PerCP/Cy5.5 (HIT3a, BioLegend). Forward (FSC) and side (SSC) light scatters were used to gate broadly on mononucleated cells, and then low SSC-W and low F SC-W gates were drawn to discriminate singlet cell events to collect CD3⁻CD19^{+CD}20⁺CD27⁺ memory B cells and CD3⁻CD19^{lo/-}CD20⁻CD27⁺+CD38⁺+ plasmablasts, which were sorted using a FACSAria Fusion cell sorter (BD Biosciences).

FIG. 8. Enzyme-linked immunosorbent assay (ELISA) against influenza antigens. Antibodies sequences from single-cell emulsion RT-PCR were cloned into an IgG expression vector and expressed in Expi293F cells. ELISA was performed using recombinantly expressed HAs from the influenza virus strains indicated.

### DESCRIPTION OF ILLUSTRATIVE EMBODIMENTS

The present disclosure generally relates to sequencing two or more genes expressed in a single cell in a high-throughput manner. More particularly, the present disclosure provides a method for high-throughput sequencing of pairs of transcripts co-expressed in single cells to determine pairs of polypeptide chains that comprise immune receptors (e.g., antibody VH and VL sequences).

The methods of the present disclosure allow for the repertoire of immune receptors and antibodies in an individual organism or population of cells to be determined. Particularly, the methods of the present disclosure may aid in determining pairs of polypeptide chains that make up immune receptors. B cells and T cells each express immune receptors; B cells express immunoglobulins, and T cells express T cell receptors (TCRs). Both types of immune receptors consist of two polypeptide chains. Immunoglobulins consist of variable heavy (VH) and variable light (VL) chains. TCRs are of two types: one consisting of an a and a β chain, and one consisting of a γ and a δ chain. Each of the polypeptides in an immune receptor has a constant region and a variable region. Variable regions result from recombination and end joint rearrangement of gene fragments on the chromosome of a B or T cell. In B cells additional diversification of variable regions occurs by somatic hypermutation. Thus, the immune system has a large repertoire of receptors, and any given receptor pair expressed by a lymphocyte is encoded by a pair of separate, unique transcripts. Only by knowing the sequence of both transcripts in the pair can the receptor as a whole be studied. Knowing the sequences of pairs of immune receptor chains expressed in a single cell is also essential to ascertaining the immune repertoire of a given individual or population of cells.

Currently available methods to analyze multiple transcripts in single cells, such as the two transcripts that comprise adaptive immune receptors, are limited by low throughput, very high instrumentation and reagent costs, and the need to capture the transcripts on a substrate. See U.S. Pat. No. 9,708,654, which is incorporated herein by reference in its entirety. No technology currently exists for rapidly analyzing how many cells express a set of transcripts of interest or, more specifically, for sequencing native lymphocyte receptor chain pairs at very high throughput (greater than 10,000 cells per run) without a capture step. The present disclosure aims to correct these deficiencies by providing a new technique for sequencing multiple transcripts simultaneously at the single-cell level with a throughput two to three orders of magnitude greater than the current state of the art.

One advantage of the methods of the present disclosure is that the methods result in a higher throughput several orders of magnitude larger than the current state of the art. In addition, the present disclosure allows for the ability to link two transcripts for large cell populations in a high throughput manner, faster and at a much lower cost than competing technologies.

In certain embodiments, the present disclosure provides methods comprising separating single cells in a compartment with oligonucleotides; lysing the cells; allowing mRNA transcripts released from the cells to hybridize with the oligonucleotides; performing overlap extension reverse transcriptase polymerase chain reaction to covalently link DNA from at least two transcripts derived from a single cell; and sequencing the linked DNA. In certain embodiments, the cells may be mammalian cells. In certain embodiments, the cells may be B cells, T cells, NKT cells, or cancer cells.

In other embodiments, the present disclosure provides methods comprising separating single cells in a compartment with oligonucleotides; lysing the cell; allowing mRNA transcripts released from the cells to hybridize with the oligonucleotides; performing reverse transcriptase polymerase chain reaction to form at least two cDNAs from at least two transcripts derived from a single cell; and sequencing the cDNA.

In other embodiments, the present disclosure provides a system comprising an aqueous fluid phase exit disposed within an annular flowing oil phase, wherein the aqueous phase fluid comprises a suspension of cells and is dispersed within the flowing oil phase, resulting in emulsified droplets with low size dispersity comprising an aqueous suspension of cells.

In other embodiments, the present disclosure provides a composition comprising an oligonucleotide capable of binding mRNA, and two or more primers specific for a transcript of interest.

In certain embodiments, the present disclosure also provides for a device comprising ordered arrays of microwells, each with dimensions designed to accommodate a single lymphocyte cell. In one embodiment, the microwells may be circular wells 56 μm in diameter and 50 μm deep, for a total volume of 125 pL. Such microwells would normally range in volume from 20-3,000 pL, though a wide variety of well sizes, shapes and dimensions may be used for single cell accommodation. In certain embodiments, the microwell may be a nanowell. In certain embodiments, the device may be a chip. The device of the present disclosure allows the direct entrapment of tens of thousands of single cells, with each cell in its own microwell, in a single chip. In certain embodiments, the chip may be the size of a microscope slide. In one embodiment, a microwell chip may be used to capture single cells in their own individual microwells. The microwell chip can be made from polydimethylsiloxane (PDMS); however, other suitable materials known in the art such as polyacrylimide, silicon and etched glass may also be used to create the microwell chip.

In certain embodiments, the oligonucleotides may be a poly(T), a sequence specific for heavy chain amplification, and/or a sequence specific for light chain amplification. A dialysis membrane covers the microwells, keeping the cells in the microwells while lysis reagents are dialyzed into the microwells. The lysis reagents cause the release of the cells' mRNA transcripts into the microwell. In embodiments where the oligonucleotide is poly(T), the poly(A) mRNA tails are captured by the poly(T) oligonucleotides. In another embodiment, the oligonucleotide may be a primer specific to a transcript of interest. The mRNA are then incubated in solution with reagents for overlap extension (OE) reverse transcriptase polymerase chain reaction (RT-PCR). This reaction mix includes primers designed to create a single PCR product comprising cDNA of two transcripts of interest covalently linked together. Before thermocycling, the reagent solution is emulsified in oil phase to create droplets. The linked cDNA products of OE RT-PCR are recovered and used as a template for nested PCR, which amplifies the linked transcripts of interest. The purified products of nested PCR are then sequenced and pairing information is analyzed. In other embodiments, restriction and ligation may be used to link cDNA of multiple transcripts of interest. In other embodiments, recombination may be used to link cDNA of multiple transcripts of interest.

The present disclosure also provides a method to trap mRNA from single cells, perform cDNA synthesis, link the sequences of two or more desired cDNAs from single cells to create a single molecule, and finally reveal the sequence of the linked transcripts by High Throughput (Next-gen) sequencing. According to the present disclosure, one way to increase throughput in biological assays is to use an emulsion that generates a high number of 3-dimensional parallelized microreactors. Emulsion protocols in molecular biology often yield 109-1011 droplets per mL (sub-pL volume). Emulsion-based methods for single-cell polymerase chain reaction (PCR) have found a wide acceptance, and emulsion PCR is a robust and reliable procedure found in many next-generating sequencing protocols. However, very high throughput RT-PCR in emulsion droplets has not yet been implemented because cell lysates within the droplet inhibit the reverse transcriptase reaction. Cell lysate inhibition of RT-PCR can be mitigated by dilution to a suitable volume.

An aqueous solution with a suspension of cells is emulsified into oil phase by injecting an aqueous cell/bead suspension into a fast-moving stream of oil phase. The shear forces generated by the moving oil phase create droplets as the aqueous suspension is injected into the stream, creating an emulsion with a low dispersity of droplet sizes. Each cell is in its own droplet. The uniformity of droplet size helps to ensure that individual droplets do not contain more than one cell. Cells are then thermally lysed, and the mixture is cooled. The mRNA is incubated in a solution for emulsion OE RT-PCR to link the cDNAs of transcripts of interest together. Nested PCR and sequencing of the linked transcripts is performed according to the present disclosure. In certain embodiments, the aqueous suspension of cells comprises reverse transcription reagents. In certain other embodiments, the aqueous suspension of cells comprises at least one of polymerase chain reaction and reverse transcriptase polymerase chain reaction reagents, including a single enzyme that is capable of catalyzing both the PCR and the RT reactions. In other embodiments, restriction and ligation may be used to link cDNA of multiple transcripts of interest. In other embodiments, recombination may be used to link cDNA of multiple transcripts of interest.

In another embodiment, emulsion droplets which contain individual cells and RT-PCR reagents are formed by injection into a fast-moving oil phase. Thermal cycling is then performed on these droplets directly. In certain embodiments, an overlap extension reverse transcription polymerase chain reaction may be used to link cDNA of multiple transcripts of interest.

Primer design for OE RT-PCR determines which transcripts of interest expressed by a given cell are linked together. For example, in certain embodiments, primers can be designed that cause the respective cDNAs from the VH and VL chain transcripts to be covalently linked together. Sequencing of the linked cDNAs reveals the VH and VL sequence pairs expressed by single cells. In other embodiments, primer sets can also be designed so that sequences of TCR pairs expressed in individual cells can be ascertained or so that it can be determined whether a population of cells co-expresses any two genes of interest.

Bias can be a significant issue in PCR reactions that use multiple amplification primers because small differences in primer efficiency generate large product disparities due to the exponential nature of PCR. One way to alleviate primer bias is by amplifying multiple genes with the same primer, which is normally not possible with a multiplex primer set. By including a common amplification region to the 5′ end of multiple unique primers of interest, the common amplification region is thereby added to the 5′ end of all PCR products during the first duplication event. Following the initial duplication event, amplification is achieved by priming only at the common region to reduce primer bias and allow the final PCR product distribution to remain representative of the original template distribution.

Such a common region can be exploited in various ways. One clear application is to add the common amplification primer at higher concentration and the unique primers (with 5′ common region) at a low concentration, such that the majority of nucleic acid amplification occurs via the common sequence for reduced amplification bias.

Accordingly, in certain embodiments, the present disclosure provides methods comprising adding a common sequence to the 5′ region of two or more oligonucleotides that are specific to a set of gene targets; and performing nucleic acid amplification of the set of gene targets by priming the common sequence.

The methods of the present disclosure allow for information regarding multiple transcripts expressed from a single cell to be obtained. In certain embodiments, probabilistic analyses may be used to identify native pairs with read counts or frequencies above non-native pair read counts or frequencies. The information may be used, for example, in studying gene co-expression patterns in different populations of cancer cells. In certain embodiments, therapies may be tailored based on the expression information obtained using the methods of the present disclosure. Other embodiments may focus on discovery of new lymphocyte receptors.

### I. ENZYMES FOR USE IN THE PRESENT EMBODIMENTS

In some embodiments, enzymes having the ability to generate DNA from a template that comprises RNA bases, either in part or in its entirety, are used. In certain embodiments, the enzymes are as described in PCT/US2017/014082, which is incorporated herein by reference in its entirety. In specific embodiments, the enzymes are recombinant enzymes. In some embodiments, the enzymes have the ability to use RNA as a template when their parent enzyme from which they were derived (by mutation) lacked such ability. In specific cases, the enzymes that acquire reverse transcriptase activity are able to recognize alternative bases or sugars in a template strand (compared to an enzyme that can only recognize DNA as a template), such as by allowing recognition of a template having uracil instead of thymine and having variability at the 2′ position in the ribose ring.

The enzymes of the present disclosure make it easier to melt RNA structure and generate cDNA copies, in specific embodiments. Although there are other commercially available reverse transcriptases with modest thermostability, the enzymes of the present disclosure have much higher thermostability (e.g., thermostability at temperatures above 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., or more) and have proofreading activity. In specific embodiments, the enzymes of the present disclosure are more processive and/or more primer-dependent, resulting in less promiscuity in generating an accurate cDNA imprint of an mRNA population, for example. Because of their proofreading domain, the enzymes of the present disclosure generate fewer mutations than other enzymes and provide a more accurate representation of the RNAs present in a given population (including, for example, a sample from one or more individuals, environments, and so forth).

At least some enzymes of the disclosure encompass proofreading activity, which may be defined herein as the ability of the enzyme to recognize an incorrect base pair, reverse its direction and excise the mismatched base, followed by insertion of the correct base. Enzymes of the disclosure may be referred to as comprising 3′-5′ exonuclease activity. Although testing a particular enzyme for proofreading activity may be achieved in a variety of ways, in specific embodiments the enzyme is tested by dideoxy-mismatch PCR that necessitates removal of a 3′ deoxy mismatch primer prior to polymerization or primer extension reactions with 3′ terminal deoxy mismatches.

Although certain enzymes of the disclosure may be characterized as reverse transcriptases, in particular aspects the enzymes can utilize DNA, RNA, modified DNA, and/or modified RNA as a template. Modified DNA and RNA may be referred to as information nucleotide-comprising polymers that can be replicated enzymatically that contain altered chemical modifications to the backbone, sugar or base. In specific cases, the modified DNA or RNA is modified at the 2′ position of a sugar of a component of the template. Particular embodiments encompass recombinant Archaeal Family-B polymerases that transcribe a template that is DNA, RNA, modified DNA, or modified RNA.

The enzymes of the disclosure may be generated using a starting polymerase that lacks reverse transcriptase activity, and in specific embodiments, that starting polymerase is an Archaeal Family-B polymerase, such as KOD polymerase. Any number of mutations may be generated from the starting polymerase and tested for using methods of the disclosure. In specific embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more mutations are incorporated into a polymerase that lacks reverse transcriptase activity such that the entirety of mutations (or a sub-combination thereof) are responsible for imparting reverse transcriptase activity to the polymerase that originally lacked it. The mutations may be of any kind, including amino acid substitution(s), deletion(s), insertion(s), inversion(s), and so forth. In specific embodiments, the mutation is a single amino acid change, and the change may or may not be conservative. Although in some cases the amino acid substitution mutation must be to a certain amino acid, in other cases the mutation may be to any amino acid. Embodiments within the scope herein are not limited by the means of generating/designing the various enzymes. While some enzymes are designed via mutations to a starting polymerase, embodiments herein are not limited to any particular mechanism of action and an understanding of the mechanism of action is not necessary to practice such embodiments.

In certain embodiments, an enzyme of the disclosure has a specific amino acid sequence identity compared to a given enzyme, for example a wild-type Archaeal Family-B polymerase, such as KOD polymerase (including, for example, SEQ ID NO:1). In specific embodiments, the enzyme has an amino acid sequence that is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identical to the amino acid sequence of SEQ ID NO:1. An enzyme of the disclosure may be of a certain length, including at least or no more than 600, 625, 650, 675, 700, 725, 750, 755, 760, 765, 770, 775, 780, 781, 782, 783, or 784 amino acids in length, for example. The enzyme may or may not be labeled. The enzyme may be further modified, such as comprising new functional groups such as phosphate, acetate, amide groups, or methyl groups, for example. The enzymes may be phosphorylated, glycosylated, lapidated, carbonylated, myristoylated, palmitoylated, isoprenylated, farnesylated, alkylated, hydroxylated, carboxylated, ubiquitinated, deamidated, contain unnatural amino acids by altered genetic codes, contain unnatural amino acids incorporated by engineered synthetase/tRNA pairs, and so forth. The skilled artisan recognizes that post-translational modification of the enzymes may be detected by one or more of a variety of techniques, including at least mass spectrometry, Eastern blotting, Western blotting, or a combination thereof, for example.

Specific examples of enzymes of the disclosure include at least the following:

(SEQ ID NO: 1)
MILDTDYITEDGKPVIRIFKKENGEFKIEYDRTFEPYFYALLKDDSAIEE
VKKITAERHGTVVTVKRVEKVQKKFLGRPVEVWKLYFTHPQDVPAIRDKI
REHPAVIDIYEYDIPFAKRYLIDKGLVPMEGDEELKMLAFDIETLYHEGE
EFAEGPILMISYADEEGARVITWKNVDLPYVDVVSTEREMIKRFLRVVKE
KDPDVLITYNGDNEDFAYLKKRCEKLGINFALGRDGSEPKIQRMGDRFAV
EVKGRIHFDLYPVIRRTINLPTYTLEAVYEAVFGQPKEKVYAEEITTAWE
TGENLERVARYSMEDAKVTYELGKEFLPMEAQLSRLIGQSLWDVSRSSTG
NLVEWFLLRKAYERNELAPNKPDEKELARRRQSYEGGYVKEPERGLWENI
VYLDFRSLYPSIIITHNVSPDTLNREGCKEYDVAPQVGHRFCKDFPGFIP
SLLGDLLEERQKIKKKMKATIDPIERKLLDYRQRAIKILANSYYGYGYAR
ARWYCKECAESVTAWGREYITMTIKEIEEKYGFKVIYSDTDGEFATIPGA
DAETVKKKAMEFLKYINAKLPGALELEYEGFYKRGFEVTKKKYAVIDEEG
KITTRGLEIVRRDWSEIAKETQARVLEALLKDGDVEKAVRIVKEVTEKLS
KYEVPPEKLVIHEQITRDLKDYKATGPHVAVAKRLAARGVKIRPGTVISY
IVLKGSGRIGDRAIPFDEFDPTKHKYDAEYYIENQVLPAVERILRAFGYR
KEDLRYQKTRQVGLSAWLKPKGT.

B11 reverse transcriptase (an example of a derivative of KOD polymerase that is a hyperthermophilic reverse transcriptase):

(SEQ ID NO: 2)
MILDTDYITEDGKPVIRIFKKENGEFKIEYDRTFEPYLYALLKDDSAIEE
VKKITAERHSTVVTVKRVEKVQKKFLGRSVEVWKLYFTHPQDVPAIMDKI
REHPAVIDIYEYDIPFAIRYLIDKGLVPMEGDEELKLLALDIGTPCHEGE
VFAEGPILMISYADEEGTRVITWRNVDLPYVDVLSTEREMIQRFLRVVKE
KDPDVLITYNGDNFDFAYLKKRCEKLGINFTLGREGSEPKIQRMGDRFAV
EVKGRIHFDLYPVIRRTVNLPIYTLEAVYEAVFGQPKEKVYAEEITTAWE
TGENLERVARYSMEDAKVTYELGKEFMPMEAQLSRLIGQSLWDVSRSSTG
NLVEWFLLRKAYERNELAPNKPDEKELARRHQSHEGGYIKEPERGLWENI
VYLDFRSLYPSIIITHNVSPDTLNREGCKEYDVAPQVGHRFCKDFPGFIP
SLLGDLLEERQKIKKRMKATIDPIERKLLDYRQRAIKILANSLYGYYGYA
RARWYCKECAESVIAWGREYITMTIKEIEEKYGFKLIYSDTDGFFATIPG
AEAETVKKKAMEFLKYINAKLPGALELEYEGFYKRGLFVTKKKYAVIDEE
GKITTRGLEIVRRDWSEIAKETQARVLEALLKDGDVEKAVRIVKEVTEKL
SKYEVPPEKLVIHKQITRDLKDYKATGPHVAVAKRLAARGVKIRPGTVIS
YIVLKGSGRIVDRAIPFDEFDPTKHKYDAEYYIENQVLPAVERILRAYGY
RKEDLWYQKTRQVGLSARLKPKGT

CORE3 reverse transcriptase (an example of a derivative of KOD polymerase that is a hyperthermophilic proofreading reverse transcriptase):

(SEQ ID NO: 3)
MILDTDYITEDGKPVIRIFKKENGEFKIEYDRTFEPYLYALLKDDSAIEE
VKKITAERHGTVVTVKRVEKVQKKFLGRPVEVWKLYFTHPQDVPAIMDKI
REHPAVIDIYEYDIPFAIRYLIDKGLVPMEGDEELKLLAFDIETLYHEGE
EFAEGPILMISYADEEGARVITWKNVDLPYVDVVSTEREMIKRFLRVVKE
KDPDVLITYNGDNFDFAYLKKRCEKLGINFALGRDGSEPKIQRMGDRFAV
EVKGRIHFDLYPVIRRTINLPTYTLEAVYEAVFGQPKEKVYAEEITTAWE
TGENLERVARYSMEDAKVTYELGKEFLPMEAQLSRLIGQSLWDVSRSSTG
NLVEWFLLRKAYERNELAPNKPDEKELARRHQSHEGGYIKEPERGLWENI
VYLDFRSLYPSIIITHNVSPDTLNREGCKEYDVAPQVGHRFCKDFPGFIP
SLLGDLLEERQKIKKRMKATIDPIERKLLDYRQRAIKILANSLYGYYGYA
RARWYCKECAESVIAWGREYLTMTIKEIEEKYGFKVIYSDTDGFFATIPG
ADAETVKKKAMEFLKYINAKLPGALELEYEGFYKRGLFVTKKKYAVIDEE
GKITTRGLEIVRRDWSEIAKETQARVLEALLKDGDVEKAVRIVKEVTEKL
SKYEVPPEKLVIHKQITRDLKDYKATGPHVAVAKRLAARGVKIRPGTVIS
YIVLKGSGRIVDRAIPFDEFDPTKHKYDAEYYIEKQVLPAVERILRAFGY
RKEDLRYQKTRQVGLSARLKPKGT

In particular aspects, the enzymes of the disclosure have one or more mutations in at least one of the following regions of a particular polymerase (here, as it corresponds to SEQ ID NO:1): residues (1-130 and 338-372 is N-terminal domain); (131-338 is exonuclease domain); (448-499 is finger domain); (591-774 is thumb domain); (374-447 and 500-590 is palm domain).

In certain embodiments, the enzymes of the disclosure have mutations at particular amino acids (the position of which corresponds to SEQ ID NO:1, in certain examples) and, in some cases particular residues are the substituted amino acid at that position. Table A provides an example of a list of certain mutations that may be present in the disclosure, and in specific embodiments a combination of mutations is utilized in the enzyme.

TABLE A
Amino acid substitutions for polymerase enzymes of the embodiments

| KOD Position | Mutation for RT activity | Possible other mutations |
|---|---|---|
| Y384 | H, I | F, L, A C, S, H, I, M, N, Q |
| V389 | I, L | M, F, T, Y, N |
| E664 | K, Q | |
| Y493 | L, C, F | I, V H T |
| R97 | Any mutation | |
| I521 | L | F, V, M, T |
| G711 | V, S, R | L, C, T, N, H, Q, K, M |
| N735 | K | R, Q, N, Y, H |
| A490 | T | V, S, C |
| F587 | L, I | A, T, V |
| M137 | L, I | A, T, V |
| K118 | I | M, V, L |
| T514 | I | V, L, M |
| R381 | H | S, Q, K |
| F38 | L, I | V, M, S |
| K466 | R | E, D, Q |
| E734 | K | R, Q, N |

In at least some cases, the enzymes have a mutation at R97 as it corresponds to SEQ ID NO:1. In some cases, two or more, three or more, four or more, five or more, six or more, seven or more, eight or more, nine or more, ten or more, eleven or more, twelve or more, thirteen or more, fourteen or more, fifteen or more, or sixteen or more mutations from this table are present in an enzyme of the disclosure. In specific embodiments, the following combinations are included alone or with one or more other mutations listed above or not listed above:

Y384 and V389; Y384 and E664; Y384 andY493; Y384 and R97; Y384 and 1521; Y384 and G711; Y384 and N735; Y384 and A490; V389 and E664; V389 and Y493; V389 and R97; V389 and 1521; V389 and G711; V389 and N735; V389 and A490; E664 and Y493; E664 and R97; E664 and 1521; E664 and G711; E664 and N735; E664 and A490; Y493 and R97; Y493 and 1521; Y493 and G711; Y493 and N735; Y493 and A490; R97 and 1521; R97 and 1521; R97 and G711; R97 and N735; R97 and A490; 1521 and G711; 1521 and N735; 1521 and A490; G711 and N735; or G711 and A490. In at least some cases, one or more other mutations are combined with these specific combinations.

In specific embodiments, the polymerase has an amino acid substitution at one or more of the following positions corresponding to SEQ ID NO:1:
a) R97; Y384; V389; Y493; F587; E664; G711; and W768;
b) F38; R97; K118; R381; Y384; V389; Y493; T514; F587; E664; G711; and W768;
c) F38; R97; K118; M137; R381; Y384; V389; K466; Y493; T514; F587; E664; G711; and W768; or
d) F38; R97; K118; M137; R381; Y384; V389; K466; Y493; T514; 1521; F587; E664; G711; N735; and W768.

Any of the combinations in a), b), c), or d) may include A490, F587, M137, K118, T514, R381, F38, K466, and/or E734. In particular embodiments, the polymerase has one or more of the following specific amino acid substitutions corresponding to SEQ ID NO:1:
a) R97M; Y384H; V389I; Y493L; F587L; E664K; G711V; and W768R;
b) F38L; R97M; K1181; R381H; Y384H; V389I; Y493L; T514I; F587L; E664K; G711V; and W768R;
c) F38L; R97M; K1181; M137L; R381H; Y384H; V389I; K466R; Y493L; T514I; F587L; E664K; G711V; and W768R; or
d) F38L; R97M; K118I; M137L; R381H; Y384H; V389I; K466R; Y493L; T514I; I521L; F587L; E664K; G711V; N735K; and W768R.

Any of the combinations in a), b), c), or d) may include A490, F587, M137, K118, T514, R381, F38, K466, and/or E734.

### II. KITS OF THE DISCLOSURE

All or some of the essential materials and reagents required for carrying out methods of the disclosure may be provided in a kit. The kit may comprise one or more of RNA base-comprising primers, DNA base-comprising primers, vectors, polymerase-encoding nucleic acids, buffers, ribonucleotides, deoxyribonucleotides, salts, and so forth corresponding to at least some embodiments of the provided methods. Embodiments of kits may comprise reagents for the detection and/or use of a control nucleic acid or enzyme, for example. Kits may provide instructions, controls, reagents, containers, and/or other materials for performing various assays or other methods (e.g., those described herein) using the enzymes of the disclosure.

The kits generally may comprise, in suitable means, distinct containers for each individual reagent, primer, and/or enzyme. In specific embodiments, the kit further comprises instructions for producing, testing, and/or using enzymes of the disclosure.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Flow-Joint Apparatus

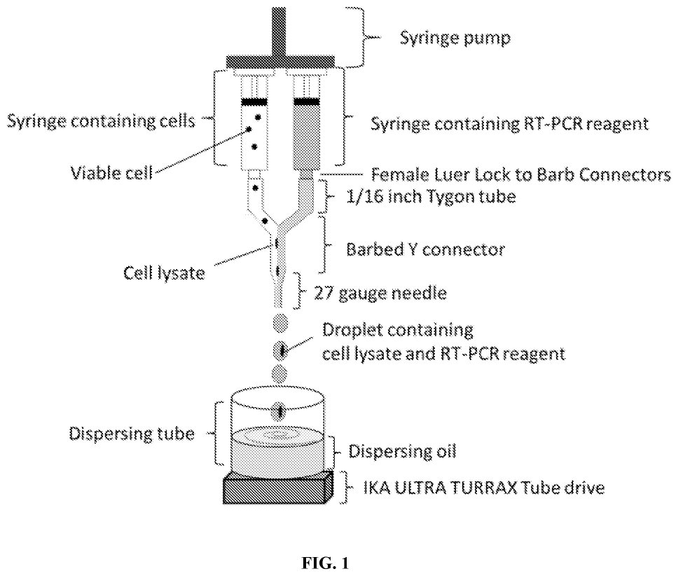

The flow-joint apparatus comprises a barbed Y connector (PVDF, 1/16″, #3063342, Cole-Parmer) that facilitates the merger of two input streams from separate 5 mL syringes into a 27-gauge needle (#Z192384-100EA, Sigma Aldrich). The syringes are connected to 1/16 inch Tygon tubing (#80-10002-03, Cytek Biosciences) via female Luer lock to barb connectors (#11532, Qosina) (FIG. 1). In a typical experiment, one syringe contains viable cells suspended in buffer, and the other contains a 2×RT-PCR solution with surfactant.

### Example 2

### Overlap Extension (OE) Emulsion RT-PCR

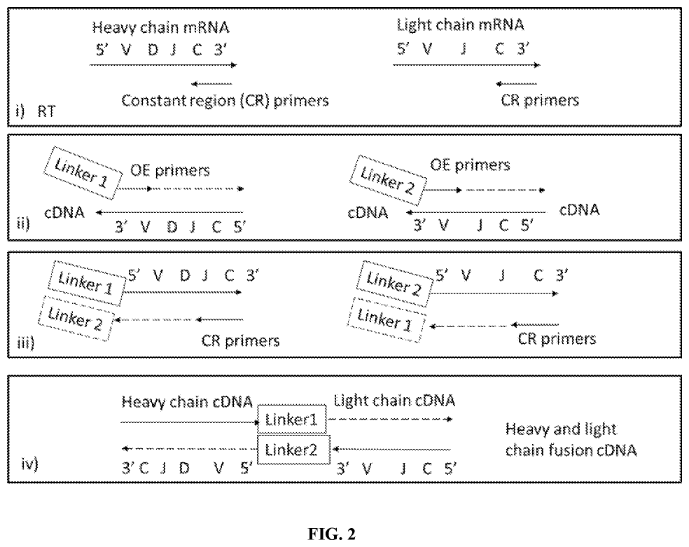

To physically link the antibody heavy and light chain transcripts from a single cell, cell lysate isolated from single cells is co-emulsified with a RT-PCR solution composed of 0.5×RTX buffer, 1.6 U/μL SUPERase.In RNase Inhibitor (Invitrogen), 0.4 mM dNTP, 2 M Betaine (Sigma-Aldrich), RTX 8 μg/mL, 0.1 wt % BSA (Invitrogen Ultrapure BSA, 50 mg/mL) and primer sets designed for overlap extension RT-PCR (Table 1). The oil phase consists of mineral oil (Sigma Aldrich Corp.) supplemented with 0.05% Triton X-100 (Sigma Aldrich Corp.) and 2% ABIL EM 90 (Degussa). The emulsions are distributed into a 96-well PCR plate and subjected to overlap-extension RT-PCR under the following conditions: 30 min at 68° C., 2 min at 94° C., followed by 25 cycles of 94° C. for 30 s, 60° C. for 30 s, and 68° C. for 2 min. Final reaction products are extended at 68° C. for 7 min (FIG. 2).

TABLE 1
Overlap Extension (OE) RT-PCR primer mix for human
antibody analysis

| | | SEQ | |
|---|---|---|---|
| Conc. | | ID | |
| (nM) | Primer ID | NO | Sequence |
| 400 | AHX89 | 4 | CGCAGTAGCGGTAAACGGC |
| 400 | BRH06 | 5 | GCGGATAACAATTTCACACAGG |
| 40 | hIgM CR | 6 | CGCAGTAGCGGTAAACGGCCGACGGGGAAT |
| | | | TCTCACAGGAGACGAGGGGGAAA |
| 40 | hIgG CR | 7 | CGCAGTAGCGGTAAACGGCGGAGSAGGGYG |
| | | | CCAGGGGGAAGAC |
| 40 | hIgA CR | 8 | CGCAGTAGCGGTAAACGGCGCTCAGCGGGA |
| | | | AGACCTTGGGGCTGG |
| 40 | hIgL CR | 9 | GCGGATAACAATTTCACACAGGTTGRAGCT |
| | | | CCTCAGAGGAGGGYGGGAA |
| 40 | hIgK CR | 10 | GCGGATAACAATTTCACACAGGCTGCTCAT |
| | | | CAGATGGCGGGAAGATGAAGACAGATGGTG |
| | | | CAG |
| 40 | hVH1-fwd-OE | 11 | TATTCCCATGGCGCGCCCAGGTCCAGCTKG |
| | | | TRCAGTCTGG |
| 40 | hVH157-fwd- | 12 | TATTCCCATGGCGCGCCCAGGTGCAGCTGG |
| | OE | | TGSARTCTGG |
| 40 | hVH2-fwd-OE | 13 | TATTCCCATGGCGCGCCCAGRTCACCTTGA |
| | | | AGGAGTCTG |
| 40 | hVH3-fwd-OE | 14 | TATTCCCATGGCGCGCCGAGGTGCAGCTGK |
| | | | TGGAGWCY |
| 40 | hVH4-fwd-OE | 15 | TATTCCCATGGCGCGCCCAGGTGCAGCTGC |
| | | | AGGAGTCSG |
| 40 | hVH4-DP63- | 16 | TATTCCCATGGCGCGCCCAGGTGCAGCTAC |
| | fwd-OE | | AGCAGTGGG |
| 40 | hVH6-fwd-OE | 17 | TATTCCCATGGCGCGCCCAGGTACAGCTGC |
| | | | AGCAGTCA |
| 40 | hVH3N-fwd- | 18 | TATTCCCATGGCGCGCCTCAACACAACGGT |
| | OE | | TCCCAGTTA |
| 40 | hVK1-fwd-OE | 19 | GGCGCGCCATGGGAATAGCCGACATCCRGD |
| | | | TGACCCAGTCTCC |
| 40 | hVK2-fwd-OE | 20 | GGCGCGCCATGGGAATAGCCGATATTGTGM |
| | | | TGACBCAGWCTCC |
| 40 | hVK3-fwd-OE | 21 | GGCGCGCCATGGGAATAGCCGAAATTGTRW |
| | | | TGACRCAGTCTCC |
| 40 | hVK5-fwd-OE | 22 | GGCGCGCCATGGGAATAGCCGAAACGACAC |
| | | | TCACGCAGTCTC |
| 40 | hVL1-fwd-OE | 23 | GGCGCGCCATGGGAATAGCCCAGTCTGTSB |
| | | | TGACGCAGCCGCC |
| 40 | hVL1459- | 24 | GGCGCGCCATGGGAATAGCCCAGCCTGTGC |
| | fwd-OE | | TGACTCARYC |
| 40 | hVL15910- | 25 | GGCGCGCCATGGGAATAGCCCAGCCWGKGC |
| | fwd-OE | | TGACTCAGCCMCC |
| 40 | hVL2-fwd-OE | 26 | GGCGCGCCATGGGAATAGCCCAGTCTGYYC |
| | | | TGAYTCAGCCT |
| 40 | hVL3-fwd-OE | 27 | GGCGCGCCATGGGAATAGCCTCCTATGWGC |
| | | | TGACWCAGCCAA |
| 40 | hVL-DPL16- | 28 | GGCGCGCCATGGGAATAGCCTCCTCTGAGC |
| | fwd-OE | | TGASTCAGGASCC |
| 40 | hVL3-38- | 29 | GGCGCGCCATGGGAATAGCCTCCTATGAGC |
| | fwd-OE | | TGAYRCAGCYACC |
| 40 | hVL6-fwd-OE | 30 | GGCGCGCCATGGGAATAGCCAATTTTATGC |
| | | | TGACTCAGCCCC |
| 40 | hVL78-fwd- | 31 | GGCGCGCCATGGGAATAGCCCAGDCTGTGG |
| | OE | | TGACYCAGGAGCC |

### Example 3

### Generation of VH:VL Fusion Amplicons Using RTX

Whether RTX and commercially available RT-PCR kits retain their polymerase activity in the emulsion containing cell lysate was investigated. Blood was drawn from a healthy female volunteer after informed consent had been obtained. PBMCs were isolated from the blood, resuspended in the RPMI-1640 containing 10% DMSO and 10% FBS, and then were frozen for cryopreservation. Total B cells were isolated from thawed PBMCs using the reagents of a Memory B Cell Isolation Kit (Miltenyi Biotec). Total B cells were washed with cold 80 mM Tris-HCl (pH7.5) twice and concentrated to 6.6×10⁸ cells/mL. One million total B cells were lysed with 100 μL following RT-PCR reagents containing surfactant. RT-PCR reagent using RTX: 1×RTX buffer (60 mM Tris-HCl (pH 8.4), 25 mM (NH₄)₂SO₄, 10 mM KCl, 1 mM MgSO₄), 0.8 U/μL SUPERase⋅In RNase Inhibitor (Invitrogen), 0.2 mM dNTPs, 1 M Betaine (Sigma-Aldrich), 0.4 μg RTX, 0.05 wt % BSA (Invitrogen Ultrapure BSA, 50 mg/mL), 0.5% Tween 20 (Sigma-Aldrich), and primer sets designed for overlap extension RT-PCR (Table 1). Three different commercially available RT-PCR reagents were used for this experiment (QIAGEN® OneStep RT-PCR Kit (QIAGEN), qScript One-Step Fast qRT-PCR Kit, ROX (Quanta Biosciences), and SuperScript™ III One-Step RT-PCR System with Platinum™ Taq DNA Polymerase (Thermo Fisher Scientific)). The RT-PCR reagents were prepared according to the manufacturer's protocol and supplemented with BSA, primers, and Tween 20 as described above. These RT-PCR reagents containing cell lysate were injected into 5.5 mL oil independently (molecular biology grade mineral oil (Sigma Aldrich Corp.) supplemented with 0.05% Triton X-100 (Sigma Aldrich Corp.) and 2% ABIL EM 90 (Degussa)) and stirred by IKA dispersing tube (DT-20, VWR) on the IKA ULTRA TURRAX Tube drive at 615 RPM for 5 min. The resulting emulsions were distributed into 96-well plates and RT-PCR was performed as follows: RT-PCR using RTX: 30 min at 68° C., 2 min at 94° C., followed by 25 cycles of 94° C. for 30 s, 60° C. for 30 s, 68° C. for 2 min. The final product was extended at 68° C. for 7 min. QIAGEN RT-PCR kit: 30 min at 55° C., 3 min at 94° C., followed by 35 cycles of 94° C. for 30 s, 60° C. for 30 s, 72° C. for 2 min. The final product was extended at 72° C. for 7 min. Quanta Biosciences RT-PCR kit: 30 min at 55° C., 2 min at 94° C., followed by 25 cycles of 94° C. for 30 s, 60° C. for 30 s, 72° C. for 2 min. The final product was extended at 72° C. for 7 min. Thermo Fisher Scientific RT-PCR kit: 30 min at 60° C., 2 min at 94° C., followed by 35 cycles of 94° C. for 30 s, 60° C. for 30 s, 68° C. for 2 min. The final product was extended at 68° C. for 7 min. As positive controls, 30 ng total B cell RNAs were mixed with RT-PCR reagents and regular RT-PCR without emulsion was performed.

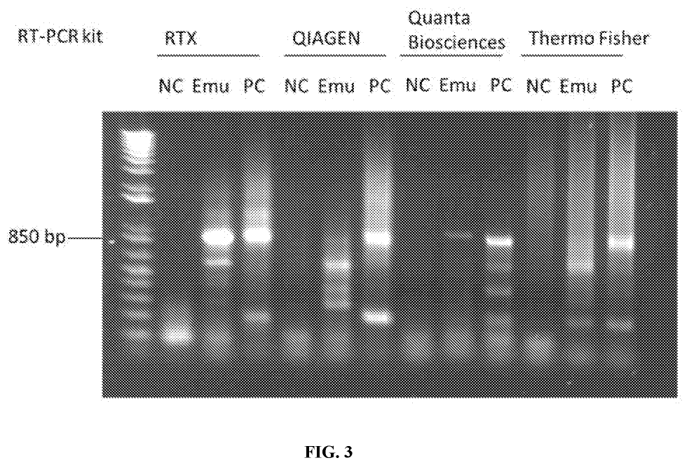

Following RT-PCR, the emulsions were collected in Eppendorf tubes and centrifuged at 17,000 g for 10 min. The mineral oil phase was decanted, and the DNA amplicons were recovered via three serial extractions using (in order) diethyl ether, water-saturated ethyl acetate, and diethyl ether. Residual ether was removed using a SpeedVac (30 minutes at RT) and the DNA was concentrated using a PCR purification kit (Zymo research Corp.) as per the manufacturer's instructions and eluted with 40 μL water. Nested PCR was performed in a total volume of 50 μL using 2 μL of the cDNA, nested primers (Table 2), and DreamTaq™ Hot Start DNA Polymerase (Thermo Fisher Scientific) according to the manufacturer's protocol and the following conditions: 95° C. for 3 min, followed by 40 cycles of 95° C. for 30 s, 62° C. for 30 s, 72° C. for 1 min. Finally, DNA was extended at 72° C. for 7 min. DNA was run on a 1% agarose gel and detected (FIG. 3).

TABLE 2
Nested PCR primers for human antibody analysis

| | | SEQ | |
|---|---|---|---|
| Conc. | | ID | |
| (nM) | Primer ID | NO | Sequence |
| 200 | Nested | 32 | NNNNATGGGCCCTGSGATGGGCCCTTGGT |
| | hIgG | | GGARGC |
| 200 | Nested | 33 | NNNNATGGGCCCTGGGTTGGGGCGGATGC |
| | hIgM | | ACTCC |
| 200 | Nested | 34 | NNNNATGGGCCCTGCTTGGGGCTGGTCGG |
| | hIgA | | GGATG |
| 200 | Nested | 35 | NNNNGTGCGGCCGCAGATGGTGCAGCCAC |
| | hIgK | | AGTTC |
| 200 | Nested | 36 | NNNNGTGCGGCCGCGAGGGYGGGAACAGA |
| | hIgL | | GTGAC |

### Example 4

### Single-Cell Emulsion RT-PCR

Blood was drawn from a healthy 36-year-old female volunteer after informed consent had been obtained. PBMCs were isolated from the blood, resuspended in RPMI-1640 containing 10% DMSO and 10% FBS, and then frozen for cryopreservation. Memory B cells were isolated from thawed PBMCs using the Memory B Cell Isolation Kit (Miltenyi Biotec). Approximately 564,000 memory B cells were obtained and cultured in RPMI-1640 medium containing 10% FBS, 2 mM L-glutamine, 1 x non-essential amino acids, 1× sodium pyruvate, and 1× penicillin/streptomycin (Life Technologies) and expanded for four days in the presence of 10 μg/mL anti-CD40 antibody (5C3, BioLegend), 1 μg/mL CpG ODN 2006 (Invivogen, San Diego, Calif., USA), 100 units/mL IL-4, 100 units/mL IL-10, and 50 ng/mL IL-21 (PeproTech, Rocky Hill, N.J., USA). Expanded B cells were washed with 15 mL 2×RTX buffer (1×RTX buffer: 60 mM Tris-HCl (pH 8.4), 25 mM (NH₄)₂SO₄, 10 mM KCl, 1 mM MgSO₄), and cell number was determined.

Two technical replicates were performed, each utilizing approximately 25,000 expanded memory B cells spiked with 300 ARH-77 cells. The cells were reconstituted in 1.4 mL 2×RTX buffer and loaded into a 5 mL syringe. Another syringe contained 1.4 mL RT-PCR solution, composed of 0.5×RTX buffer, 1.6 U/μL SUPERase⋅In RNase Inhibitor (Invitrogen), 0.4 mM dNTPs, 2 M Betaine (Sigma-Aldrich), RTX 8μg/mL, 0.1 wt % BSA (Invitrogen Ultrapure BSA, 50 mg/mL), 0.5% (v/v) Tween 20 (Sigma-Aldrich), and primer sets designed for overlap extension RT-PCR (Table 1). Both syringes were simultaneously compressed by a syringe pump (KD Scientific Legato 200, Holliston, Mass., USA) at the speed of 1.3 mL/min, and the resulting stream was directly injected into 9 mL of chilled oil (molecular biology grade mineral oil (Sigma Aldrich Corp.) supplemented with 0.05% Triton X-100 (Sigma Aldrich Corp.) and 2% ABIL EM 90 (Degussa)) stirred by IKA dispersing tube (DT-20, VWR) on the IKA ULTRA TURRAX Tube drive at 615 RPM (FIG. 1). Five minutes following emulsification, the resulting emulsions were aliquoted into 96-well PCR plates and subjected to overlap-extension RT-PCR under the following conditions: 30 min at 68° C., 2 min at 94° C., followed by 25 cycles of 94° C. for 30 s, 60° C. for 30 s, 68° C. for 2 min. The final product was extended at 68° C. for 7 min.

Following RT-PCR, the emulsions were collected in Eppendorf tubes and centrifuged at 17,000 g for 10 min. The mineral oil phase was decanted, and the DNA amplicons were recovered via three serial extractions using (in order) diethyl ether, water-saturated ethyl acetate, and diethyl ether. Residual ether was removed using a SpeedVac (30 minutes at RT) and the DNA was concentrated using a PCR purification kit (Zymo research Corp.) as per the manufacturer's instructions. Nested PCR was performed in a total volume of 250 μL using 100 ng cDNA, nested primers (Table 2), and Platinum™ Taq DNA Polymerase (Thermo Fisher Scientific) according to the manufacturer's protocol and the following conditions: 94° C. for 3 min, followed by 25 cycles of 94° C. for 30 s, 62° C. for 30 s, 72° C. for 30 s. Finally, DNA was extended at 72° C. for 7 min. The 850 bp PCR product was isolated from a 1% agarose gel using a gel purification kit (Zymo Research Corp.) according to the manufacturer's protocol.

A two-step procedure was performed to append Illumina adaptor sequences to the amplicon. First, 50 ng of DNA was amplified using NEBNext® High-Fidelity 2×PCR Master Mix (New England BioLabs Inc) in combination with the primers in Table 3 under the following conditions: 98° C. for 30 s, followed by 8 cycles of 98° C. for 10 s, 62° C. for 30 s, 72° C. for 30 s, and finally a 7 min extension at 72° C. The PCR product was concentrated using a PCR purification kit and quantified by Nanodrop. In the second reaction, 50 ng of DNA was amplified by NEBNext® High-Fidelity 2×PCR Master Mix in combination with the primers in Table 4 under the following conditions: 98° C. for 30 s, followed by 8 cycles of 98° C. for 10 s, 62° C. for 30 s, 72° C. for 30 s, and finally a 7 min extension at 72° C. The 1100 bp PCR product was isolated from a 1% agarose gel using a gel isolation kit and submitted for Illumina MiSeq 2×300 sequencing.

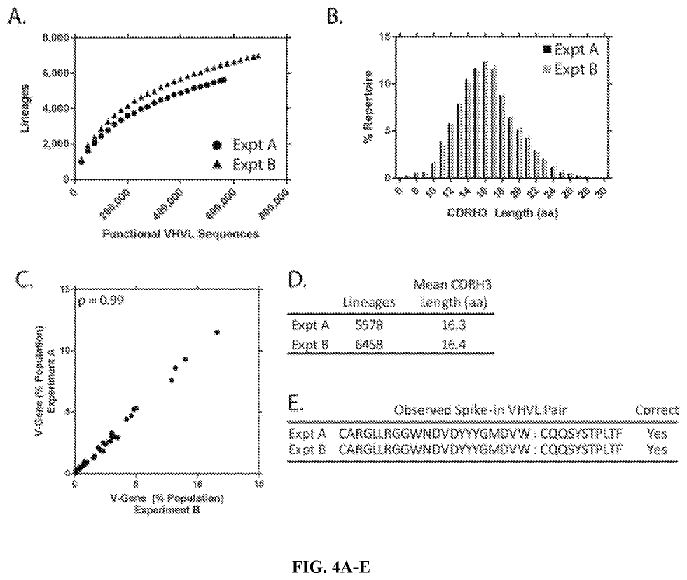

Raw 2×300 Illumina reads were trimmed and filtered to remove low quality sequences using Trimmomatic and submitted to MiXCR for CDR3 identification and gene annotation. Sequences with >2 reads were grouped into lineages based on 90% CDRH3 nucleotide identity using Usearch (version 7.0). Rarefaction analysis was performed by subsampling the raw Illumina reads to measure the sample diversity independent from the number of sequencing reads (FIG. 4A). Two independent technical replicates analyzing 25,000 cells each yielded 5,578 and 6,458 lineages, thereby exhibiting a minimum efficiency range of 22-25% (assuming no clonal expansion). To examine reproducibility, the dominant sequence in each lineage by read count was used to calculate the distribution of CDRH3 lengths (FIG. 4B, 4D) and gene usage (FIG. 4C). CDRH3 lengths matched the typical human repertoire, suggesting that this technique does not significantly impact the observed CDRH3 length. The absolute frequency of V-genes was also highly consistent across both experiments (ρ=0.99, Spearman correlation). To determine pairing fidelity, the sample was spiked with 300 ARH-77 cells (1.2% of total). The spike-in cell line was observed in both experiments with the correct VH:VL pair (FIG. 4E).

TABLE 3
PCR primers for adding Illumina adaptor sequences

| Conc. | Primer | SEQ ID | |
|---|---|---|---|
| (nM) | ID | NO | Sequence |
| 100O | hIgG_MiSeqRev | 37 | GTCTCGTGGGCTCGGAGATGTGTA |
| | | | TAAGAGACAGNNNNATGGGCCCTG |
| | | | SGATGGGCCCTTGGTGGARGC |
| 1000 | hIgM_MiSeqRev | 38 | GTCTCGTGGGCTCGGAGATGTGTA |
| | | | TAAGAGACAGNNNNATGGGCCCTG |
| | | | GGTTGGGGCGGATGCACTCC |
| 1000 | hIgA_MiSeqRev | 39 | GTCTCGTGGGCTCGGAGATGTGTA |
| | | | TAAGAGACAGNNNNATGGGCCCTG |
| | | | CTTGGGGCTGGTCGGGGATG |
| 1000 | hIgK_MiSeqRev | 40 | TCGTCGGCAGCGTCAGATGTGTAT |
| | | | AAGAGACAGNNNNGTGCGGCCGCA |
| | | | GATGGTGCAGCCACAGTTC |
| 1000 | hIgL_MiSeqRev | 41 | TCGTCGGCAGCGTCAGATGTGTAT |
| | | | AAGAGACAGNNNNGTGCGGCCGCG |
| | | | AGGGYGGGAACAGAGTGAC |

TABLE 4
PCR primers for adding Illumina adaptor sequences

| Conc. | Primer | SEQ ID | |
|---|---|---|---|
| (nM) | ID | NO | Sequence |
| 1000 | MiSeqFw | 42 | AATGATACGGCGACCACCGAGATCTACAC |
| | | | GACGACTCGTCGGCAGCGTC |
| 1000 | MiSeqRev1 | 43 | CAAGCAGAAGACGGCATACGAGATGCCTAA |
| | | | GTCTCGTGGGCTCGG |
| 1000 | MiSeqRev2 | 44 | CAAGCAGAAGACGGCATACGAGATTGGTCA |
| | | | GTCTCGTGGGCTCGG |

### Example 5

### Generation of PGK1 cDNA Using RTX

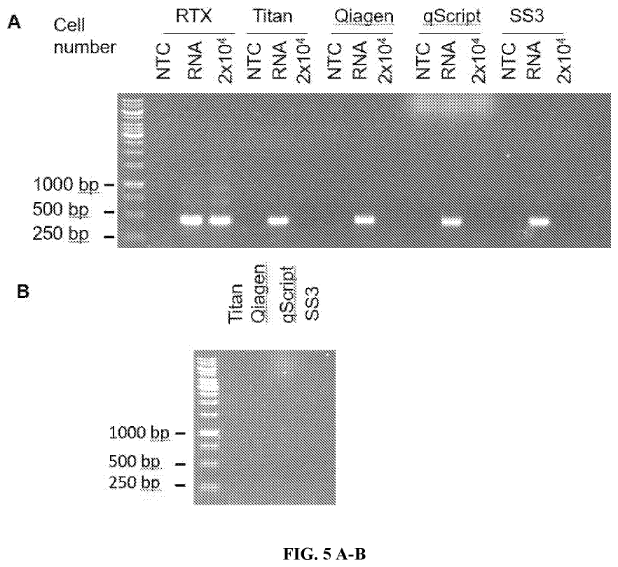

HEK293 cells were gently dissociated from the culturing plate by pipetting and centrifuged at 300×g. The culture medium was removed, cells were resuspended in cold 1 mL 80 mM Tris-HCl (pH 7.5) and then centrifuged at 900×g for 5 min. The supernatant was removed and this washing step was repeated. The cells were resuspended in the cold 80 mM Tris-HCl (pH 7.5) at the concentration of 100,000 cells/μL and then 0.2 μL cell suspension was mixed with the 50 μl various RT-PCR reagents (RTX, Titan One Tube RT-PCR System (#11855476001, Sigma), QIAGEN® OneStep RT-PCR Kit (#210210, QIAGEN), SuperScript® III One-Step RT-PCR System (#12574-026, ThermoFisher Scientific), qScript One-Step Fast qRT-PCR Kit, ROX (#95080-500, Quanta Biosciences)) containing 0.5% Tween 20. The RT-PCR reagent recipes are described in Table 5. 300 ng total RNA from HEK293 cells was used as a positive control. The PGK1 primer sequences are described in Table 6. RT-PCR to detect PGK1 mRNA was performed as follows: RT-PCR using RTX: 30 min at 68° C., 2 min at 94° C., followed by 25 cycles of 94° C. for 30 s, 60° C. for 30 s, 68° C. for 1 min. The final product was extended at 68° C. for 7 min. Titan One Tube RT-PCR System: 30 min at 50° C., 2 min at 94° C., followed by 35 cycles of 94° C. for 30 s, 60° C. for 30 s, 68° C. for 1 min. The final product was extended at 72° C. for 7 min. QIAGEN RT-PCR kit: 30 min at 50° C., 5 min at 95° C., followed by 35 cycles of 94° C. for 30 s, 60° C. for 30 s, 72° C. for 1 min. The final product was extended at 72° C. for 7 min. Quanta Biosciences RT-PCR kit: 30 min at 55° C., 2 min at 94° C., followed by 35 cycles of 94° C. for 30 s, 60° C. for 30 s, 72° C. for 1 min. The final product was extended at 72° C. for 7 min. Thermo Fisher Scientific RT-PCR kit: 30 min at 60° C., 2 min at 94° C., followed by 35 cycles of 94° C. for 30 s, 60° C. for 30 s, 68° C. for 1 min. The final product was extended at 68° C. for 7 min. The resulting DNAs were run on a 1% agarose gel and detected (FIG. 5A). Since other one-pot emulsion RT-PCR studies employed two minutes 65° C. initial heating step to lyse the cells (Turchaninova et al., 2013; Mitchell et al., 2017; and Munson et al., 2016, each incorporated herein by reference), it was tested whether this initial heating step would improve the RT-PCR results. However, PGK1 cDNA could not be obtained with the heat lysing in our condition (FIG. 5B).

TABLE 5
RT-PCR recipe for PGK1 amplification

| RTX | Volume (μL) |
|---|---|
| 10x RTX buffer | 5 |
| 5M Betaine (#B0300-5VL, Sigma) | 10 |
| 10 mM dNTP (#N0447L, New England BioLabs) | 1 |
| 10 μM PGK1 forward primer | 1 |
| 10 μM PGK1 reverse primer | 1 |
| SUPERase• In ™ RNase Inhibitor | 2.5 |
| (#AM2696, ThermoFisher) | |
| Tween 20 (#P9416, Sigma) | 0.25 |
| 0.2 μg/μL RTX enzyme | 1 |
| Ultrapure water | to 50 μL |
| Titan One Tube RT-PCR System | |
| (#11855476001, Sigma) | Volume (μL) |
| 5x RT-PCR Buffer | 10 |
| 10 μM PGK1 forward primer | 1 |
| 10 μM PGK1 reverse primer | 1 |
| Enzyme mix | 1 |
| SUPERase• In ™ RNase Inhibitor | 2.5 |
| (#AM2696,ThermoFisher) | |
| 10 mM dNTP (#N0447L, New England BioLabs) | 1 |
| 100 mM DTT solution | 2.5 |
| Tween20 | 0.25 |
| Ultrapure water | to 50 μL |
| QIAGENO ® OneStep RT-PCR Kit (#210210, QIAGEN) | Volume (μL) |
| 5x QIAGEN RT-PCR buffer | 10 |
| 10 mM dNTP (#N0447L, New England BioLabs) | 1 |
| 10 μM PGK1 forward primer | 1 |
| 10 μM PGK1 reverse primer | 1 |
| SUPERase• In ™ RNase Inhibitor | 2.5 |
| (#AM2696, ThermoFisher) | |
| Tween 20 (#P9416, Sigma) | 0.25 |
| One Step RT-PCR Enzyme Mix | 2 |
| Ultrapure water | to 50 μL |
| qScript One-Step qRT-PCR Kit | |
| (#95080-500, Quanta Biosciences) | Volume (μL) |
| One-Step Fast Master Mix, ROX (4X) | 12.5 |
| One-Step Fast RT | 2.5 |
| 10 μM PGK1 forward primer | 1 |
| 10 μM PGK1 reverse primer | 1 |
| SUPERase• In ™ RNase Inhibitor | 2.5 |
| (#AM2696, ThermoFisher) | |
| Tween 20 (#P9416, Sigma) | 0.25 |
| Ultrapure water | to 50 μL |
| SuperScript ® III One-Step RT-PCR System | |
| (#12574-026, ThermoFisher Scientific) | Volume (μL) |
| 2x Reaction mix | 25 |
| 10 μM PGK1 forward primer | 1 |
| 10 μM PGK1 reverse primer | 1 |
| SUPERase• In ™ RNase Inhibitor | 2.5 |
| (#AM2696,ThermoFisher) | |
| Tween 20 (#P9416, Sigma) | 0.25 |
| SuperScript ™ III RT/Platinum ™ Taq Mix | 2 |
| Ultrapure water | to 50 μL |

TABLE 6
RT-PCR primers for PGK1 mRNA amplification

| Conc. | | SEQ | |
|---|---|---|---|
| (nM) | Primer ID | ID NO | Sequence |
| 200 | PGK1 Fw | 45 | AGGTGCTCAACAACATGGAGA |
| 200 | PGK1 Rev | 46 | CCCCAGTGCTCACATGGCTGACTTT |

### Example 6

### Single-Cell Emulsion RT-PCR (BCR Pairing Using Different B Cells)

VH-VL pairing accuracy and throughput was examined using expanded human B cells. Frozen PBMCs from a healthy 36-year-old female volunteer (Table 7, Donor A, same donor as in Example 4) were thawed and CD27⁺ memory B cells were isolated by a Memory B Cell Isolation Kit (Miltenyi Biotec) and expanded for four days as described in Example 4. The expanded memory B cells were divided into two replicates. Each replicate contained 30,000 expanded B cells and 500 ARH-77 B cells were added as a spike-in control (60:1 ratio). Single-cell emulsion RT-PCR was performed as described in Example 4 and with the volumes described in Table 7. The resulting VH-VL amplicons were purified as described in Example 4. Nested PCR was performed in a total volume of 250 μL using 30% volume of the cDNA, nested primers (Table 2), and DreamTaq™ Hot Start DNA Polymerase (Thermo Fisher Scientific) according to the manufacturer's protocol and the following conditions: 95° C. for 3 min, followed by 28 cycles of 95° C. for 30 s, 62° C. for 30 s, 72° C. for 1 min. Finally, DNA was extended at 72° C. for 7 min. DNA was run on a 1% agarose gel and detected. The 850 bp PCR product was isolated from a 1% agarose gel using a gel purification kit (Zymo Research Corp.) according to the manufacturer's protocol. The Illumina adaptor sequences were added as described in Example 4 and with the MiSeqFw primer in Table 4 and MiSeqRev3 (IgGA, sample A), MiSeqRev4(IgM, sample A), MiSeqRev5 (IgGA, sample A′), or MiSeqRev6 (IgM, sample A′) in Table 8.

TABLE 7
Analysis of paired inmiune receptor repertoire using single-cell emulsion RT.PCR

| | | | Cell | Buffer | | | | | | Vol- | |
|---|---|---|---|---|---|---|---|---|---|---|---|
| | | | concentration | volume | Number of | | | | | ume | Dis- |
| | Sample | | in a syringe | in a syringe | BCR/TCR | Pairing | | Do- | | of oil | persing |
| | name | Cell number | (cells/mL) | (mL) | clusters | precision | SuperaseIN | nor | Oil | (mL) | tube |
| Replicate | A | 3 × 10⁴ expanded | 1.6 × 10⁴ | 1.83 | VH-VL: 5,761 | 93.8% | + | A | ABIL | 11.3 | DT 20 |
| | | CD27⁺ B cells + | | | | | | | EM 90 | | |
| | | 500 ARH-77 | | | | | | | based | | |
| | A′ | 3 × 10⁴ expanded | 1.6 × 10⁴ | 1.83 | VH-VL: 5,260 | | + | | oil | 11.3 | |
| | | CD27⁺ B cells | | | | | | | | | |
| | | 500 ARH-77 | | | | | | | | | |
| Replicate | B | 1.83 × 10⁵ expanded | 1.0 × 10⁵ | 1.83 | VH-VL: 21,801 | 96.5% | + | | | 11.3 | |
| | | CD27⁺ B cells + | | | | | | | | | |
| | | 500 ARH-77 | | | | | | | | | |
| | B′ | 1.83 × 10⁵ expanded | 1.0 × 10⁵ | 1.83 | VH-VL: 17,223 | | − | | | 11.3 | |
| | | CD27⁺ B cells + | | | | | | | | | |
| | | 500 ARH-77 | | | | | | | | | |
| Replicate | C | 1.45 × 10⁵ expanded | 7.9 × 10⁴ | 1.83 | TCRαβ: 6,186 | 93.4% | + | B | Span 80 | 11.3 | |
| | | pan T cells | | | | | | | based | | |
| | C′ | 1.45 × 10⁵ expanded | 7.9 × 10⁴ | 1.83 | TCRαβ: 7,023 | | + | | oil | 11.3 | |
| | | pan T cells | | | | | | | | | |
| Replicate | D | 3.62 × 10⁵ | 2.0 × 10⁵ | 1.81 | TCRαβ: 12,736 | 92.9% | − | A | ABIL | 11.2 | |
| | | expanded pan T cells | | | | | | | EM 90 | | |
| | D′ | 3.62 × 10⁵ | 2.0 × 10⁵ | 1.81 | TCRαβ: 13,811 | | − | | based | | |
| | | expanded pan T cells | | | | | | | oil | | |
| | E | 1.0 × 10⁶ PBMCs | 2.0 × 10⁵ | 5 | VH-VL: 3,276 | N.A | + | C | | 31 | DT 50 |
| Replicate | F | 6.5 × 10⁵ PBMCs | 1.3 × 10⁵ | 5 | TCRαβ: 7,064 | 90.2% | + | | | 31 | |
| | F' | 6.5 × 10⁵ PBMCs + | 1.3 × 10⁵ | 5 | TCRαβ: 7,325 | | − | | | 31 | |
| | | 103 Jurkat | | | | | | | | | |

TABLE 8
PCR primers for adding IIlumina adaptor sequences

| | | SEQ | |
|---|---|---|---|
| Conc. | | ID | |
| (nM) | Primer ID | NO | Sequence |
| 1000 | MiSeqRev3 | 47 | CAAGCAGAAGACGGCATACGAGAT ACATCG GTCTCGTGGGCTCGG |
| 1000 | MiSeqRev4 | 48 | CAAGCAGAAGACGGCATACGAGAT TCAAGT GTCTCGTGGGCTCGG |
| 1000 | MiSeqRev5 | 49 | CAAGCAGAAGACGGCATACGAGAT CTGATC GTCTCGTGGGCTCGG |
| 1000 | MiSeqRev6 | 50 | CAAGCAGAAGACGGCATACGAGAT TACAAG GTCTCGTGGGCTCGG |
| 1000 | MiSeqRev7 | 51 | CAAGCAGAAGACGGCATACGAGAT TTTCAC GTCTCGTGGGCTCGG |
| 1000 | MiSeqRev8 | 52 | CAAGCAGAAGACGGCATACGAGAT AGGAAT GTCTCGTGGGCTCGG |
| 1000 | MiSeqRev9 | 53 | CAAGCAGAAGACGGCATACGAGAT GGAACT GTCTCGTGGGCTCGG |
| 1000 | MiSeqRev10 | 54 | CAAGCAGAAGACGGCATACGAGAT GCTCAT GTCTCGTGGGCTCGG |
| 1000 | MiSeqRev11 | 55 | CAAGCAGAAGACGGCATACGAGAT CCACTC GTCTCGTGGGCTCGG |
| 1000 | MiSeqRev12 | 56 | CAAGCAGAAGACGGCATACGAGAT AACTTC GTCTCGTGGGCTCGG |

DNA was sequenced using Illumina MiSeq 2×300. 5,761 VH-VL clusters in sample A and 5,260 VH-VL clusters in sample A′ (Table 7) were detected. Among both replicates, 3,166 identical CDR-H3 amino acid sequences were observed, which must have been originated from identical B cell progenitors. Out of the identical CDR-H3 sequences, 2,786 CDR-H3 paired with identical CDR-L3 in both replicates. This results in 93.8% pairing precision (Table 7, see the formula below for the pairing precision calculation). In the MiXCR annotated sequences before clustering, ARH-77 VH and VL were correctly paired and detected as 15 reads and 11 reads in sample A and sample A′, respectively. ARH-77 VH paired with incorrect VL was detected as single reads and thus were filtered out through the bioinformatic pipeline (DeKosky et al., In-depth determination and analysis of the human paired heavy- and light-chain antibody repertoire. Nature Medicine. (2015)). During the CD27⁺ memory B cell isolation step with the kit, CD27⁻ B cells were also isolated, which mostly represent naïve B cells. CD27⁻ B cells were expanded using the same protocol. 1.83×10⁵ expanded B cells were mixed with 500 ARH-77 cells (366:1 ratio) and performed single-cell emulsion RT-PCR. A technical replicate experiment was performed without SUPERase⋅In™ RNase inhibitor. The resulting VH-VL amplicons were analyzed as described in Example 4. For sequencing, MiSeqFw primer in Table 4 and MiSeqRev7 (IgGA, sample B), MiSeqRev8 (IgM, sample B), MiSeqRev9 (IgGA, sample B′), or MiSeqRev10 (IgM, sample B′) in Table 8 were used for adding Illumina adaptor sequences. 21,801VH-VL clusters in sample B and 17,223 VH-VL clusters in sample B′ (Table 7) were detected. Among both replicates, 4,976 identical CDR-H3 amino acid sequences were observed, which must have been originated from identical B cell progenitors. Out of the identical CDR-H3 sequences, 4,642 CDR-H3 paired with identical CDR-L3 in both replicates. This results in 96.5% pairing precision.

In the MixCR annotated sequences before clustering, the correct ARH77 VH-VL pair was detected as 118 reads in sample B and 435 reads in sample B′. In sample B, the top correct ARH-77 VH which paired with incorrect VL was detected as single reads and thus were filtered out through our bioinformatic pipeline. In sample B′, the top correct ARH-77 VH which paired with incorrect VL was detected as two reads. Thus, the signal to noise ratio in this experiment was 217.5:1 (DeKosky et al., In-depth determination and analysis of the human paired heavy- and light-chain antibody repertoire. Nature Medicine. (2015)).

The pairing precision was calculated with the following formula as described before (DeKosky et al., 2015; McDaniel et al., 2016).

$$P = \sqrt{\frac{{TP}_{1 and 2}}{{TP}_{1 and 2} + {FP}_{1 or 2}}}$$

TP1 and 2 is the number of VH sequences paired with identical VL sequences in both replicates. FP1 or 2 is the number of VH sequences paired with different VL sequences across the replicates. P is the VH-VL pairing precision. To estimate the TCR pairing precision, VH was replaced with TCRβ and VL was replaced with TCRα.

### Example 7

### Single-Cell Emulsion RT-PCR (TCR Pairing)

Next, it was tested whether the methods could be used to analyze paired TCRαβ at the single-cell level by the single-cell emulsion RT-PCR. Blood was drawn from a healthy 59-year-old female volunteer (Donor B, Table 7) after informed consent had been obtained. PBMCs were isolated from the blood, resuspended in the RPMI-1640 containing 10% DMSO and 10% FBS, and then were frozen for cryopreservation. The frozen PBMCs were thawed and total T cells were isolated with Pan T cell isolation kit (#130-096-535, Miltenyi Biotec). The T cells were cultured in RPMI-1640 medium containing 10% FBS, 2 mM L-glutamine, 1× non-essential amino acids, 1× sodium pyruvate, and 1× penicillin/streptomycin (Life Technologies) and expanded in the presence of CD3/CD28 dynabeads (#11161D, Thermo Fisher Scientific) and 30 units/mL IL-2 (PeproTech) for a week. The medium was exchanged every three days and fresh beads and IL-2 were added. 2.9×10⁵ expanded T cells were divided into two replicates. Single-cell emulsion RT-PCR was performed for each replicate as described in Example 4 but using the primers described in Table 9 to pair TCRαβ. In this experiment, Span80 based oil (mineral oil containing 4.5% Span-80(#56760, Sigma Aldrich), 0.4% Tween 80(#P9416, Sigma Aldrich), 0.05% Triton X-100, v/v%) was used. The volumes of reagents were described in the Table 7. The TCRα and TCRβ primers are the modification of the following reference. (Han et al., 2014).

TABLE 9
Overlap Extension (OE) RT-PCR primer mix for human TCRαβ analysis

| | | | | Primer |
|---|---|---|---|---|
| Conc. | | SEQ ID | | mixture |
| (nM) | Primer ID | NO | Sequence | name |
| 40 | TRAV1 OE | 57 | TATTCCCATGGCGCGCC | TRAV TRBV |
| | | | CTGCACGTACCAGACATCTGGGTT | OE mix |
| 40 | TRAV2 OE | 58 | TATTCCCATGGCGCGCC | |
| | | | GGCTCAAAGCCTTCTCAGCAGG | |
| 40 | TRAV3 OE | 59 | TATTCCCATGGCGCGCC | |
| | | | GGATAACCTGGTTAAAGGCAGCTA | |
| 40 | TRAV4.1 OE | 60 | TATTCCCATGGCGCGCC | |
| | | | GGATACAAGACAAAAGTTACAAACGA | |
| 40 | TRAV5 OE | 61 | TATTCCCATGGCGCGCC | |
| | | | GCTGACGTATATTTTTTCAAATATGGA | |
| 40 | TRAV6 OE | 62 | TATTCCCATGGCGCGCC | |
| | | | GGAAGAGGCCCTGTTTTCTTGCT | |
| 40 | TRAV7 OE | 63 | TATTCCCATGGCGCGCC | |
| | | | GCTGGATATGAGAAGCAGAAAGGA | |
| 40 | TRAV8 OE | 64 | TATTCCCATGGCGCGCC | |
| | | | AGGACTCCAGCTTCTCCTGAAGTA | |
| 40 | TRAV9 OE | 65 | TATTCCCATGGCGCGCC | |
| | | | GTATGTCCAATATCCTGGAGAAGGT | |
| 40 | TRAV10 OE | 66 | TATTCCCATGGCGCGCC | |
| | | | CAGTGAGAACACAAAGTCGAACGG | |
| 40 | TRAV12.1 OE | 67 | TATTCCCATGGCGCGCC | |
| | | | CCTAAGTTGCTGATGTCCGTATAC | |
| 40 | TRAV12.2 OE | 68 | TATTCCCATGGCGCGCC | |
| | | | GGGAAAAGCCCTGAGTTGATAATGT | |
| 40 | TRAV12.3 OE | 69 | TATTCCCATGGCGCGCC | |
| | | | GCTGATGTACACATACTCCAGTGG | |
| 40 | TRAV13.1 OE | 70 | TATTCCCATGGCGCGCC | |
| | | | CCCTTGGTATAAGCAAGAACTTGG | |
| 40 | TRAV13.2 OE | 71 | TATTCCCATGGCGCGCC | |
| | | | CCTCAATTCATTATAGACATTCGTTC | |
| 40 | TRAV14/DV4 OE | 72 | TATTCCCATGGCGCGCC | |
| | | | GCAAAATGCAACAGAAGGTCGCTA | |
| 40 | TRAV16 OE | 73 | TATTCCCATGGCGCGCC | |
| | | | TAGAGAGAGCATCAAAGGCTTCAC | |
| 40 | TRAV17 OE | 74 | TATTCCCATGGCGCGCC | |
| | | | CGTTCAAATGAAAGAGAGAAACACAG | |
| 40 | TRAV18 OE | 75 | TATTCCCATGGCGCGCC | |
| | | | CCTGAAAAGTTCAGAAAACCAGGAG | |
| 40 | TRAV19 OE | 76 | TATTCCCATGGCGCGCC | |
| | | | GGTCGGTATTCTTGGAACTTCCAG | |
| 40 | TRAV20 OE | 77 | TATTCCCATGGCGCGCC | |
| | | | GCTGGGGAAGAAAAGGAGAAAGAAA | |
| 40 | TRAV21 OE | 78 | TATTCCCATGGCGCGCC | |
| | | | GTCAGAGAGAGCAAACAAGTGGAA | |
| 40 | TRAV22 OE | 79 | TATTCCCATGGCGCGCC | |
| | | | GGACAAAACAGAATGGAAGATTAAGC | |
| 40 | TRAV23/DV6 OE | 80 | TATTCCCATGGCGCGCC | |
| | | | CCAGATGTGAGTGAAAAGAAAGAAG | |
| 40 | TRAV24 OE | 81 | TATTCCCATGGCGCGCC | |
| | | | GACTTTAAATGGGGATGAAAAGAAGA | |
| 40 | TRAV25 OE | 82 | TATTCCCATGGCGCGCC | |
| | | | GGAGAAGTGAAGAAGCAGAAAAGAC | |
| 40 | TRAV26.1 OE | 83 | TATTCCCATGGCGCGCC | |
| | | | CCAATGAAATGGCCTCTCTGATCA | |
| 40 | TRAV26.2 OE | 84 | TATTCCCATGGCGCGCC | |
| | | | GCAATGTGAACAACAGAATGGCCT | |
| 40 | TRAV27 OE | 85 | TATTCCCATGGCGCGCC | |
| | | | GGTGGAGAAGTGAAGAAGCTGAAG | |
| 40 | TRAV29/DV5 OE | 86 | TATTCCCATGGCGCGCC | |
| | | | GGATAAAAATGAAGATGGAAGATTCAC | |
| 40 | TRAV30 OE | 87 | TATTCCCATGGCGCGCC | |
| | | | CCTGATGATATTACTGAAGGGTGGA | |
| 40 | TRAV34 OE | 88 | TATTCCCATGGCGCGCC | |
| | | | GGTGGGGAAGAGAAAAGTCATGAA | |
| 40 | TRAV35 OE | 89 | TATTCCCATGGCGCGCC | |
| | | | GGTGAATTGACCTCAAATGGAAGAC | |
| 40 | TRAV36/DV7 OE | 90 | TATTCCCATGGCGCGCC | |
| | | | GCTAACTTCAAGTGGAATTGAAAAGA | |
| 40 | TRAV38-2/DV8 OE | 91 | TATTCCCATGGCGCGCC | |
| | | | GAAGCTTATAAGCAACAGAATGCAAC | |
| 40 | TRAV39 OE | 92 | TATTCCCATGGCGCGCC | |
| | | | GGAGCAGTGAAGCAGGAGGGAC | |
| 40 | TRAV40 OE | 93 | TATTCCCATGGCGCGCC | |
| | | | GAGAGACAATGGAAAACAGCAAAAAC | |
| 40 | TRAV41 OE | 94 | TATTCCCATGGCGCGCC | |
| | | | GCTGAGCTCAGGGAAGAAGAAGC | |
| 40 | TRBV2 OE | 95 | GGCGCGCCATGGGAATA | |
| | | | CTGAAATATTCGATGATCAATTCTCAG | |
| 40 | TRBV3-1 | 96 | GGCGCGCCATGGGAATA | |
| | | | TCATTATAAATGAAACAGTTCCAAATCG | |
| 40 | TRBV4 OE | 97 | GGCGCGCCATGGGAATA | |
| | | | AGTGTGCCAAGTCGCTTCTCAC | |
| 40 | TRBV5-4,8 OE | 98 | GGCGCGCCATGGGAATA | |
| | | | CAGAGGAAACTYCCCTCCTAGATT | |
| 40 | TRBV5-1 OE | 99 | GGCGCGCCATGGGAATA | |
| | | | GAGACACAGAGAAACAAAGGAAACTTC | |
| 40 | TRBV6-1 OE | 100 | GGCGCGCCATGGGAATA | |
| | | | GGTACCACTGACAAAGGAGAAGTCC | |
| 40 | TRBV6-2,3 OE | 101 | GGCGCGCCATGGGAATA | |
| | | | GAGGGTACAACTGCCAAAGGAGAGGT | |
| 40 | TRBV6-4 OE | 102 | GGCGCGCCATGGGAATA | |
| | | | GGCAAAGGAGAAGTCCCTGATGGTT | |
| 40 | TRBV6-5,6 OE | 103 | GGCGCGCCATGGGAATA | |
| | | | AAGGAGAAGTCCCSAATGGCTACAA | |
| 40 | TRBV6-8 OE | 104 | GGCGCGCCATGGGAATA | |
| | | | CTGACAAAGAAGTCCCCAATGGCTAC | |
| 40 | TRBV6-9 OE | 105 | GGCGCGCCATGGGAATA | |
| | | | CACTGACAAAGGAGAAGTCCCCGAT | |
| 40 | TRBV7-2 OE | 106 | GGCGCGCCATGGGAATA | |
| | | | AGACAAATCAGGGCTGCCCAGTGA | |
| 40 | TRBV7-3 OE | 107 | GGCGCGCCATGGGAATA | |
| | | | GACTCAGGGCTGCCCAACGAT | |
| 40 | TRBV7-8 OE | 108 | GGCGCGCCATGGGAATA | |
| | | | CCAGAATGAAGCTCAACTAGACAA | |
| 40 | TRBV7-4,6 OE | 109 | GGCGCGCCATGGGAATA | |
| | | | GGTTCTCTGCAGAGAGGCCTGAG | |
| 40 | TRBV7-7 OE | 110 | GGCGCGCCATGGGAATA | |
| | | | GGCTGCCCAGTGATCGGTTCTC | |
| 40 | TRBV7-9 OE | 111 | GGCGCGCCATGGGAATA | |
| | | | GACTTACTTCCAGAATGAAGCTCAACT | |
| 40 | TRBV9 OE | 112 | GGCGCGCCATGGGAATA | |
| | | | GAGCAAAAGGAAACATTCTTGAACGATT | |
| 40 | TRBV10-1,3 OE | 113 | GGCGCGCCATGGGAATA | |
| | | | GGCTRATCCATTACTCATATGGTGTT | |
| 40 | TRBV10-2 OE | 114 | GGCGCGCCATGGGAATA | |
| | | | GATAAAGGAGAAGTCCCCGATGGCT | |
| 40 | TRBV11 OE | 115 | GGCGCGCCATGGGAATA | |
| | | | GATTCACAGTTGCCTAAGGATCGAT | |
| 40 | TRBV12-3 OE | 116 | GGCGCGCCATGGGAATA | |
| | | | GATTCAGGGATGCCCGAGGATCG | |
| 40 | TRBV12-5 OE | 117 | GGCGCGCCATGGGAATA | |
| | | | GATTCGGGGATGCCGAAGGATCG | |
| 40 | TRBV13 OE | 118 | GGCGCGCCATGGGAATA | |
| | | | GCAGAGCGATAAAGGAAGCATCCCT | |
| 40 | TRBV14 OE | 119 | GGCGCGCCATGGGAATA | |
| | | | TCCGGTATGCCCAACAATCGATTCT | |
| 40 | TRBV15 OE | 120 | GGCGCGCCATGGGAATA | |
| | | | GATTTTAACAATGAAGCAGACACCCCT | |
| 40 | TRBV16 OE | 121 | GGCGCGCCATGGGAATA | |
| | | | GATGAAACAGGTATGCCCAAGGAAAG | |
| 40 | TRBV18 OE | 122 | GGCGCGCCATGGGAATA | |
| | | | TATCATAGATGAGTCAGGAATGCCAAAG | |
| 40 | TRBV19 OE | 123 | GGCGCGCCATGGGAATA | |
| | | | GACTTTCAGAAAGGAGATATAGCTGAA | |
| 40 | TRBV20-1 | 124 | GGCGCGCCATGGGAATA | |
| | | | CAAGGCCACATACGAGCAAGGCGTC | |
| 40 | TRBV24-1 OE | 125 | GGCGCGCCATGGGAATA | |
| | | | CAAAGATATAAACAAAGGAGAGATCTCT | |
| 40 | TRBV25-1 OE | 126 | GGCGCGCCATGGGAATA | |
| | | | AGAGAAGGGAGATCTTTCCTCTGAGT | |
| 40 | TRBV27-1 OE | 127 | GGCGCGCCATGGGAATA | |
| | | | GACTGATAAGGGAGATGTTCCTGAAG | |
| 40 | TRBV28 OE | 128 | GGCGCGCCATGGGAATA | |
| | | | GGCTGATCTATTTCTCATATGATGTTAA | |
| 40 | TRBV29 OE | 129 | GGCGCGCCATGGGAATA | |
| | | | GCCACATATGAGAGTGGATTTGTCATT | |
| 40 | TRBV30 OE | 130 | GGCGCGCCATGGGAATA | |
| | | | GGTGCCCCAGAATCTCTCAGCCT | |
| 200 | TRBC rev | 131 | ACCAGTGTGGCCTTTTGGGTGTGGGAG | TRAC TRBC |
| 200 | TRAC rev | 132 | CGGTGAATAGGCAGACAGACTTGTCACTGG | mix |

Following RT-PCR, the emulsions were collected in Eppendorf tubes and centrifuged at 17,000 g for 10 min. The mineral oil phase was decanted, and the DNA amplicons were recovered using two serial extractions using water-saturated diethyl ether. Residual ether was removed using a SpeedVac (30 minutes at RT) and the DNA was concentrated using a PCR purification kit (Zymo research Corp.) as per the manufacturer's instructions. For TCR analysis, eluted cDNA and AMPure XP beads (#A63880, Beckman Coulter) were mixed at a ratio of 2:1 to remove small unlinked cDNAs. After 5 min incubation, the supernatant was removed by using a magnetic rack, and the beads were washed with 200 μL 80% EtOH twice without resuspension. After 10 min drying, beads were reconstituted with 50 μL ultrapure water and the supernatant was recovered by using the magnetic rack. Nested PCR was performed in a total volume of 250 μL, using 10% volume of cDNA, nested primers (Table 10), and DreamTaq™ Hot Start DNA Polymerase (Thermo Fisher Scientific) according to the manufacturer's protocol and the following conditions: 95° C. for 3 min, followed by 30 cycles of 95° C. for 30 s, 62° C. for 30 s, 72° C. for 1 min. Finally, DNA was extended at 72° C. for 7 min. DNA was run on a 1% agarose gel and detected. The ˜550 bp PCR product was isolated from a 1% agarose gel using a gel purification kit (Zymo Research Corp.) according to the manufacturer's protocol.

TABLE 10
Nested Primers for human TCRαβ analysis

| | | SEQ | |
|---|---|---|---|
| Conc. | | ID | |
| (nM) | Primer ID | NO | Sequence |
| 200 | TRAC_Nested_4N | 133 | GTCTCGTGGGCTCGGAGATGTGTAT |
| | | | AAGAGACAGNNNN TACACGGCAG |
| | | | GGTCAGGGTT C |
| 200 | TRBC_Nested_4N | 134 | TCGTCGGCAGCGTCAGATGTGTATA |
| | | | AGAGACAGNNNN ATGGCTCAAA |
| | | | CACAGCGACC |

A one-step procedure was performed to append Illumina adaptor sequences to the amplicon. First, 50 ng of DNA was amplified using NEBNext® High-Fidelity 2×PCR Master Mix (New England BioLabs Inc) in combination with a MiSeqFw primer in Table 4 and MiSeqRev10 (sample C) or MiSeqRev11(sample C′) in Table 8 under the following conditions: 98° C. for 30 s, followed by 6 cycles of 98° C. for 10 s, 62° C. for 30 s, 72° C. for 30 s, and finally a 7 min extension at 72° C. The ˜600 bp PCR product was isolated from a 1% agarose gel using a gel isolation kit and submitted for Illumina MiSeq 2×300 sequencing.

The TCR sequences were quality filtered and annotated using the MiXCR software. Because somatic hypermutation does not occur in TCR genes, the sequences were clustered at the 97% CDR-β3 nucleotide similarity using Usearch (Dekosky et al. 2016), and TCR clusters observed by two or more reads were extract, 6,186 TCRαβ clusters were observed in sample C, and 7,023 TCRαβ clusters in sample C′. Among both replicates, 3,102 identical CDR-β3 amino acid sequences were observed, which must have been originated from identical T cell progenitors. Out of the identical CDR-β3 sequences, 2,706 CDR-β3 paired with identical CDR-α3 in both replicates. This results in 93.4% TCRαβ pairing precision (Table 7).

### Example 8

### Single-Cell Emulsion RT-PCR (TCR Pairing Using Highly Concentrated T Cells)

Next, it was tested whether cell concentration affects the pairing precision of TCRαβ. Frozen PBMCs from a healthy donor (Donor A) were thawed and total T cells were isolated by Pan T Cell Isolation Kit. The T cells were expanded for a week as described above and used for single-cell emulsion RT-PCR at the concentration 2.0×10⁵ cells/mL in a syringe. The volumes of the reagents were described in Table 7. The resulting TCRαβ cDNAs were amplified as described above. MiSeqFw primer in Table 4 and MiSeqRev5 (sample D) or MiSeqRev6 (sample D′) in Table 8 were used for adding Illumina adaptor sequence. The DNA was sequenced with Illumina MiSeq 2× 300. 13,273.5 TCRαβ clusters were detected on the average. Among both replicates, 8,746 identical CDR-β3 amino acid sequences were observed. Out of the identical CDR-β3 sequences, 7,562 CDR-β3 paired with identical CDR-α3 in both replicates. This results in 92.9% TCRαβ pairing precision (Table 7). Thus, more concentrated cells did not disrupt the throughput and pairing precision of single-cell emulsion RT-PCR. Much more concentrated cells could likely be used for single-cell emulsion RT-PCR.

### Example 9

### Single-Cell Emulsion RT-PCR for the Analysis of Vaccine-Elicited Immune Receptors

Single-cell emulsion RT-PCR to analyze immune receptors elicited by influenza vaccination. A healthy 25-year-old donor (Donor C) was vaccinated with Fluzone® Quadrivalent inactivated influenza vaccine (after informed consent had been obtained), and then PBMCs were isolated seven days after the vaccination. One million PBMCs were directly used for single-cell emulsion RT-PCR to generate VH-VL fusion amplicons in the volume described in Table 7. In parallel, 650,000 PBMCs were stimulated with 100 ng/mL PMA (#P8139, Sigma Aldrich) and 100 ng/mL ionomycin (#I9657, Sigma Aldrich) for four hours and performed single-cell emulsion RT-PCR to generate TCRαβ fusion amplicons. A technical replicate experiment for TCR sequencing was also performed without SUPERase⋅In™ RNase inhibitor. In this experiment, 1,000 Jurkat T cells were mixed with 650,000 PMA/ionomycin stimulated PBMCs and then performed single-cell emulsion RT-PCR. For these experiments, DT-50 tubes were used for the emulsification (#0003699600, IKA). The emulsion was collected and the aqueous phase were extracted using diethyl ether/ethyl acetate as described above. Then, the aqueous phase was mixed with 2.5 volumes of 100% EtOH and 0.04 volume of 3M sodium acetate and then centrifuged at 17,000×g for 30 min at 4° C. After removing the supernatant, 1 mL 70% EtOH was added and centrifuged at 17,000×g for 5 min. After removing the supernatant, the pellet was dissolved with 400 μL ultrapure water and column concentration was performed according to the manufacturer's protocol (#C1003-50, #D4004-1-L, #D4003-2-48, Zymo Research Corp). cDNA was eluted with 50 μL ultrapure water. For TCR analysis, eluted cDNA and AMPure XP beads (#A63880, Beckman Coulter) were mixed at a ratio of 2:1, and small unlinked cDNAs were removed as described above. Nested PCR was performed with DreamTaq™ Hot Start DNA Polymerase (#EP1702, ThermoFisher Scientific), primers described in Table 2 for BCR, primers described in Table 10 for TCR, 30% of cDNA for BCR, 10% of cDNA for TCR, and the following conditions: 94° C. for 3min initial denaturation, followed by 30 cycles of PCR amplification: 94° C. for 30 s, 62° C. for 30s, 72° C. forlmin. Final extension: 72° C. for 7 min. The amplicon was gel purified and Illumina adaptor sequences were added as described above. MiSeqRev12 (IgM, sample E), MiSeqRev2 (IgG, sample E), MiSeqRev2 (sample F), MiSeqRev7 (sample F′) and MiSeqFw primer were the primers used (Table4 and Table8). VH-VL and TCRαβ sequences were obtained using Illumina MiSeq 2×300 sequencing. 3,276 VH-VL clusters (Table 7, sample E), 7,064 TCRαβ clusters (Table 7, sample F) and 7,325 TCRαβ clusters (Table 7, sample F′) were detected. The TCRαβ pairing precision calculated between F and F′ was 90.2%. The top correct Jurkat-encoded TCRαβ was detected as 821 read counts whereas top Jurkat TCRβ paired with incorrect TCRα was detected as 3 read counts. Thus, the signal to noise ratio in this experiment was 273.6:1.

### Example 10

### Analysis of Vaccine Elicited Antibodies

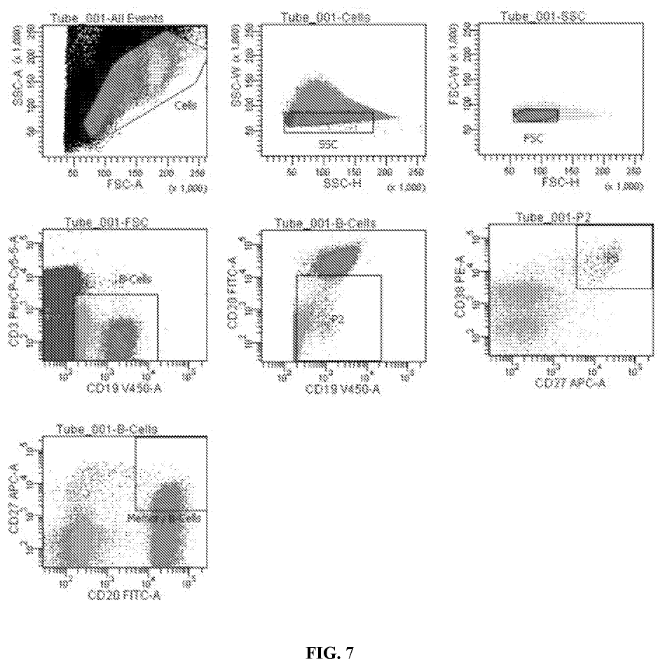

To determine antigen-specific antibody sequences, VH sequences of plasmablasts and memory B cells from the Fluzone-vaccinated donor were analyzed. The PBMCs freshly drawn from the Fluzone® vaccinee were stained at 4° C. for 15 min in PBS/0.2% BSA with anti-human CD19-v450 (HIB19, BD Biosciences, San Jose, Calif.), CD27-APC (M-T271, BD Biosciences), CD38-PE (HIT2, BioLegend, San Diego, Calif.), CD2O-FITC (2H7, BioLegend), and CD3-PerCP/Cy5.5 (HIT3a, BioLegend). Cells were washed and filtered. Forward (F SC) and side (SSC) light scatters were used to gate broadly on mononucleated cells, and then low SSC-W and low FSC-W gates were drawn to discriminate singlet cell events to collect CD3⁻CD19^{lo/-}CD20⁺CD27⁺ memory B cells and CD3⁻CD19^{lo/-}CD20⁻CD27⁺+CD38⁺+ plasmablasts, which were sorted directly into 1 mL TRIzol reagent (Thermo Fisher Scientific) using a FACSAria Fusion cell sorter (BD Biosciences) (FIG. 7). FACS sorted cells were lysed in TRIzol reagent and mixed with chloroform. After 10 min 12,000×g centrifugation at 4° C., the aqueous phase was purified using RNeasy Mini Kit (#74104, Qiagen). Plasmablasts 500 ng RNA, and memory B cell 500 ng RNA were reverse transcribed with oligo d(T)20 primer and SUPERSCRIPT® IV FIRST-STRAND SYNTHESIS SYSTEM (#18091050, Thermo Fisher Scientific), according to the manufacturer's instructions. VH cDNA was amplified with primers described in Table 11, FastStart High Fidelity PCR System (#4738292001, Sigma Aldrich) and PCR condition described in Table 12.

TABLE 11
Primers for VH sequencing

| | SEQ ID | |
|---|---|---|
| Primer ID | NO | SEQUENCE (5′ --> 3′) |
| VH1-fwd | 135 | CAGGTCCAGCTKGTRCAGTCTGG |
| VH157-fwd | 136 | CAGGTGCAGCTGGTGSARTCTGG |
| VH2-fwd | 137 | CAGRTCACCTTGAAGGAGTCTG |
| VH3-fwd | 138 | GAGGTGCAGCTGKTGGAGWCY |
| VH4-fwd | 139 | CAGGTGCAGCTGCAGGAGTCSG |
| VH4-DP63- | 140 | CAGGTGCAGCTACAGCAGTGGG |
| fwd | | |
| VH6-fwd | 141 | CAGGTACAGCTGCAGCAGTCA |
| VH3N-fwd | 142 | TCAACACAACGGTTCCCAGTTA |
| IgM-rev | 143 | GGTTGGGGCGGATGCACTCC |
| IgG-all-rev | 144 | SGATGGGCCCTTGGTGGARGC |
| IgA-all-rev | 145 | GGCTCCTGGGGGAAGAAGCC |

TABLE 12
PCR protocol for VH amplification

| 95° C. 2 min | 1 hold |
|---|---|
| 92° C. 30 s | 4 cycles |
| 50° C. 30 s | |
| 72° C. 30 s | |
| 92° C. 30 s | 4 cycles |
| 55° C. 30 s | |
| 72° C. 30 s | |
| 92° C. 30 s | 22 cycles |
| 63° C. 30 s | |
| 72° C. 30 s | |
| 72° C. 7 min | 1 hold |
| 4° C. hold | |

The resulting PCR product was isolated from a 1% agarose gel using a gel purification kit (Zymo Research Corp.) and then sequenced with Illumina MiSeq 2×300. To identify VH-VL sequences of plasmablasts or memory B cells, VH sequences from the plasmablasts and memory B cells were clustered with VH-VL sequences of sample E at the 90% CDR-H3 nucleotide similarity. To know the entire light chain sequence of the identified clonotypes, 50 ng nested PCR product of VH-VL was amplified with hIgK_MiSeqRev, hIgL_MiSeqRev (Table 2), and a primer in Table 13, NEBNext® High-Fidelity 2×PCR Master Mix (New England BioLabs Inc) under the following conditions: 98° C. for 30 s, followed by 12 cycles of 98° C. for 10 s, 62° C. for 30 s, 72° C. for 30 s, and finally a 7 min extension at 72° C. The product was column purified and eluted with 30 μL ultrapure water. Then Illumina adaptor sequence was introduced to the product as described above by using MiSeqRev3(Table 8) and MiSeqFw (Table 4) primers. The product was sequenced with Illumina MiSeq 2×300.

TABLE 13
PCR primers for adding Illumina adaptor sequences

| | | SEQ | |
|---|---|---|---|
| onc. | | ID | |
| (nM) | Primer ID | NO | Sequence |
| 1000 | Linker_VL_MiSqRev1_ | 146 | GTCTCGTGGGCTCGGAGATGTG |
| | 4N | | TATAAGAGACAGNNNN GCGCC |
| | | | GCGATGGGAAT |

Selected VH:VL sequences from plasmablasts/memory B cells (Table 14) were synthesized as gBlocks (Integrated DNA Technologies) and cloned into IgG expression vector (pcDNA3.4, Invitrogen). Heavy chain plasmid and light chain plasmid were transfected into Expi293 cells at a 1:3 ratio and the cells were incubated at 37° C. with 8% CO2 for a week. The supernatant was recovered and then mixed with 0.04 volume of 25×PBS. Subsequently, the supernatant was centrifuged at 500 g for 10 min at RT. The supernatant was passed over a column containing 1 mL Protein G agarose resin (Thermo Scientific) three times. The column was washed with 20 mL of PBS and then antibodies were eluted with 5 mL 100 mM glycine-HCl (pH 2.7), and neutralized with 1 ml 1 M Tris-HCl (pH 8.0) immediately. Antibodies were buffer-exchanged into PBS using Amicon Ultra-30 centrifugal spin columns (Millipore) and used for Enzyme-linked immunosorbent assay (ELISA).

TABLE 14
Cloned antibody sequences

| | | | | | | SEQ |
|---|---|---|---|---|---|---|
| | | | | | Full Length amino | ID |
| Clonotype | V gene | D gene | J gene | Isotype | acid sequence | NO |
| HT-A | IGHV5- | IGHD4- | IGHJ3 | IGHG1 | EVQLVESGAEVKKPGESLRISC | 147 |
| | 51 | 23 | | | EGSGYSFTSYWISWVRQMPG | |
| | | | | | KGLEWMGRIDPSDSYTNYGPS | |
| | | | | | FQGHVTISVDKSISTAYLQWN | |
| | | | | | SLKASDTAMYYCARPGGVTRD | |
| | | | | | DAFDIWGQGTMVTVSS | |
| | IGKV1- | | IGKJ2 | IGKC | DIRVTQSPSSLSASVGDRVTIT | 148 |
| | 39 | | | | CRASQSISGYLNWYQQKPGRPPK | |
| | | | | | LLIYGASSLQSGVPSRFSGSGSG | |
| | | | | | TDFTLTISSLQPEDFATY | |
| | | | | | YCQQSYGTPGNFGQGTKLEIK | |
| HT-B | IGHV4- | IGHD6- | IGHJ4 | IGHG3 | QVQLQESGPGLVKPSQTLSLT | 149 |
| | 31 | 19 | | | CTVSGDSITSGYYHWTWIR | |
| | | | | | QHPGKGLEWIGYIYYSGSTDY | |
| | | | | | NPSLKSRVIMSVDRSKNQF | |
| | | | | | SLKLHSVTAADTAVYYCERGR | |
| | | | | | PVAGTSPYFDSWGRGILVTVSS | |
| | IGLV1- | | IGLJ3 | IGLC2 | QSVLTQPPSVSGAPGQRVTI | 150 |
| | 40 | | | | SCTGSSSNIGADYDVHWYQHLP | |
| | | | | | GTAPKLLIYVSSNRPSGVPDRF | |
| | | | | | SGSKSGTSASLAITGLQAEDEAT | |
| | | | | | YYCQSYDNTLSGSEVFGGGTKLTVL | |
| HT-C | IGHV3- | IGHD6- | IGHJ6 | IGHG1 | QVQLVESGGGVVQPGTSL | 151 |
| | 30 | 25 | | | RLSCAVSGFTFSSYAMHW | |
| | | | | | VRQAPGKGLEWVAVISHD | |
| | | | | | GSSTYSPDSVKGRFTISRVIS | |
| | | | | | KNTVFLQMNSLRVEDTAV | |
| | | | | | YYCAKDFLSAAISYGMDVW | |
| | | | | | GQGTTVAVSS | |
| | IGLV3- | | IGLJ2 | IGLC7 | SYELTQPPSVSVSPGQTARIT | 152 |
| | 25 | | | | CSGEALPNQYAYWYRQKPGQAP | |
| | | | | | VLVIYKDTERPSGIPERFSGSS | |
| | | | | | SRTAVTLTISGVQAEDEADYYCQ | |
| | | | | | SPHTSGTYVIFGGGTKLTVL | |
| HT-D | IGHV4- | IGHD1- | IGHJ2 | IGHG1 | QVQLQESGPGLVRPSQTLSLTC | 153 |
| | 31 | 26 | | | TVSGDSVSSGGYSWNWIRQHP | |
| | | | | | GKGLEWIGNIPYIGSANYNPSLK | |
| | | | | | SRVSMSLDTSQNKFSLNLNFV | |
| | | | | | TAADTAVYYCARDRGSYSRYFD | |
| | | | | | LWGRGALVTVSS | |
| | IGKV1- | | IGKJ4 | IGKC | DIRVTQSPTSVSASVGDRVTITCR | 154 |
| | 12 | | | | ASQYISRRLAWYQQRPGQA | |
| | | | | | PKLLINAASSLQSGVPSRFSGSGS | |
| | | | | | DRDFTLTIRSLEPEDSATYICQ | |
| | | | | | QADSFPLTFGGGTNVHVK | |
| HT-E | IGHV3- | IGHD3-3 | IGHJ3 | IGHG1 | QVQLVESGGGLVKPGGSLRLSC | 155 |
| | 11 | | | | AASGFNFNDYYMTWIRQAPG | |
| | | | | | KGLEWLAYISGRTSFTKYADSVK | |
| | | | | | GRFTISKDNAKKTLSLQMNT | |
| | | | | | VRAEDTAVYYCGRLGDFWSGS | |
| | | | | | ESLDIWGQGTVVTVSP | |
| | IGLV1- | | IGLJ3 | IGLC7 | QPVLTQPPSASGTPGQRVVIS | 156 |
| | 44 | | | | CTGAKSNIGTNTVNWYQQFPGT | |
| | | | | | APKLLIYNNDQRPSGVPDRFSGS | |
| | | | | | RSGTSGSLAISGLQSEDEADY | |
| | | | | | HCATWDDSVNGPVFGGGTKLTVL | |

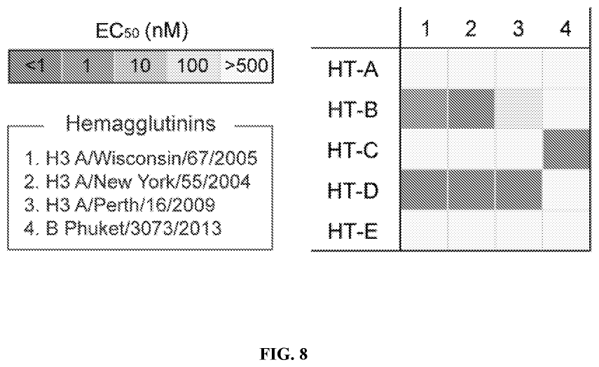

ELISA was performed with the following influenza Hemagglutinin antigens. Hemagglutinin Protein from Influenza Virus, B/Phuket/3073/2013; H3 Hemagglutinin Protein from Influenza Virus, A/Wisconsin/67/2005 (H3N2), Recombinant from Baculovirus, (#NR-15171, BEI Resources); H3 Hemagglutinin Protein from Influenza Virus, A/New York/55/2004 (H3N2), Recombinant from Baculovirus, (#NR-19241, BEI Resources); H3 Hemagglutinin Protein with C-Terminal Histidine Tag from Influenza Virus, A/Perth/16/2009 (H3N2), Recombinant from Baculovirus (#NR-42974, BEI Resources).The 50% effective concentration (EC50) values based on ELISA were used to determine the apparent binding affinities of the recombinant monoclonal antibodies. First, costar 96-well ELISA plates (Corning) were coated overnight at 4° C. with 4 μg/ml recombinant HAs and washed and blocked with 2% milk in PBS for two hours at RT. After blocking, serially diluted recombinant antibodies bound to the plates for one hour, followed by 1:5000 diluted goat anti-human IgG Fc HRP-conjugated secondary antibodies (Jackson ImmunoResearch; 109-035-008) for one hour. For detection, 50 μl TMB-ultra substrate (Thermo Scientific) was added before quenching with 50 μl M H2504. Absorbance was measured at 450 nm using a Tecan M200 plate reader. Data were analyzed and fitted for EC50 using a 4-parameter logistic nonlinear regression model in the GraphPad Prism software. All ELISA assays were performed in triplicate. As a result, three antibodies showed binding to HA antigens with high affinity (FIG. 8).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

European Patent No. EP 1 317 539 B
Aird, D. et al. Analyzing and minimizing PCR amplification bias in ILLUMINA® sequencing libraries. Genome Biol. 12, R18 (2011).
Baltimore, D. RNA-dependent DNA polymerase in virions of RNA tumour viruses. Nature 226, 1209-1211 (1970).
Bergen, K., Betz, K., Welte, W., Diederichs, K. & Marx, A. Structures of KOD and 9° N DNA Polymerases Complexed with Primer Template Duplex. ChemBioChem 14, 1058-1062 (2013).
Boeke, J. D. & Stoye, J. P. in Retroviruses (eds. Coffin, J. M., Hughes, S. H. & Varmus, H. E.) (Cold Spring Harbor Laboratory Press, 1997). at available on the world wide web at ncbi.nlm.nih.gov/books/NBK19468/>
Brochet, X., Lefranc, M.-P. & Giudicelli, V. IMGT/V-QUEST: the highly customized and integrated system for IG and TR standardized V-J and V-D-J sequence analysis. Nucleic Acids Res. 36, W503-W508 (2008).
Chan, M. et al. Evaluation of Nanofluidics Technology for High-Throughput SNP Genotyping in a Clinical Setting. J Mol Diagn 13, 305-312 (2011).
Citri, A. et al. Comprehensive qPCR profiling of gene expression in single neuronal cells. Nature Protocols 7, 118-127 (2012).
Cozens, C., Pinheiro, V. B., Vaisman, A., Woodgate, R. & Holliger, P. A short adaptive path from DNA to RNA polymerases. Proc. Natl. Acad. Sci. 109, 8067-8072 (2012).
DeKosky, B.J. et al. High-throughput sequencing of the paired human immunoglobulin heavy and light chain repertoire. Nat Biotech 31, 166-169 (2013).
DeKosky, B. J. et al. In-depth determination and analysis of the human paired heavy- and light-chain antibody repertoire. Nat. Med. 21, 86-91 (2015).
DeKosky et al., Large-scale sequence and structural comparisons of human naive and antigen-experienced antibody repertoires. Proc. Nat. Acad. Sci. (2016).
DeLuca, D. S. et al. RNA-SeQC: RNA-seq metrics for quality control and process optimization. Bioinforma. Oxf. Engl. 28, 1530-1532 (2012).
Edgar, R. C. Search and clustering orders of magnitude faster than BLAST. Bioinformatics 26, 2460-2461 (2010).
Eigen, M. Selforganization of matter and the evolution of biological macromolecules. Naturwissenschaften 58, 465-523 (1971).
Firbank, S. J., Wardle, J., Heslop, P., Lewis, R. J. & Connolly, B. A. Uracil Recognition in Archaeal DNA Polymerases Captured by X-ray Crystallography. J. Mol. Biol. 381, 529-539 (2008).
Friguet, B., Chaffotte, A.F., Djavadi-Ohaniance, L. & Goldberg, M.E. Measurements of the true affinity constant in solution of antigen-antibody complexes by enzyme-linked immunosorbent assay. Journal of Immunological Methods 77,305-319 (1985).
Fogg, M. J., Pearl, L. H. & Connolly, B. A. Structural basis for uracil recognition by archaeal family B DNA polymerases. Nat. Struct. Biol. 9, 922-927 (2002).
Ghadessy, F. J., Ong, J. L. & Holliger, P. Directed evolution of polymerase function by compartmentalized self-replication. Proc. Natl. Acad. Sci. 98, 4552-4557 (2001).
Greagg, M. A. et al. A read-ahead function in archaeal DNA polymerases detects promutagenic template-strand uracil. Proc. Natl. Acad. Sci. U. S. A. 96, 9045-9050 (1999).
Han, A., Glanville, J., Hansmann, L. & Davis, M. M. Linking T-cell receptor sequence to functional phenotype at the single-cell level. Nat. Biotechnol. 32,684-692 (2014).
Hansen, K. D., Brenner, S. E. & Dudoit, S. Biases in ILLUMINA® transcriptome sequencing caused by random hexamer priming. Nucleic Acids Res. 38, e131-e131 (2010).
Killelea, T. et al. Probing the Interaction of Archaeal DNA Polymerases with Deaminated Bases Using X-ray Crystallography and Non-Hydrogen Bonding Isosteric Base Analogues. Biochemistry (Mosc.) 49, 5772-5781 (2010).
Kim, T. W., Delaney, J. C., Essigmann, J. M. & Kool, E. T. Probing the active site tightness of DNA polymerase in subangstrom increments. Proc. Natl. Acad. Sci. U. S. A. 102, 15803-15808 (2005).
Klarmann, G. J Schauber, C. A. & Preston, B. D. Template-directed pausing of DNA synthesis by HIV-1 reverse transcriptase during polymerization of HIV-1 sequences in vitro. J. Biol. Chem. 268, 9793-9802 (1993).
Kojima, T. et al. PCR amplification from single DNA molecules on magnetic beads in emulsion: application for high-throughput screening of transcription factor targets. Nucleic Acids Res. 33 (2005).
Krause, J.C. et al. Epitope-Specific Human Influenza Antibody Repertoires Diversify by B Cell Intraclonal Sequence Divergence and Interclonal Convergence. The Journal of Immunology 187, 3704-3711 (2011).
Kyu, S.Y. et al. Frequencies of human influenza-specific antibody secreting cells or plasmablasts post vaccination from fresh and frozen peripheral blood mononuclear cells. Journal of Immunological Methods 340, 42-47 (2009).
Lauring, A. S. & Andino, R. Quasispecies Theory and the Behavior of RNA Viruses. PLoS Pathog. 6, e1001005 (2010).
Li, H. Aligning sequence reads, clone sequences and assembly contigs with BWA-MEM; alignment algorithm online at the arXiv website of Cornell University Library. (2013).
Lundberg, K. S. et al. High-fidelity amplification using a thermostable DNA polymerase isolated from Pyrococcus furiosus. Gene 108, 1-6 (1991).
Mar, J. C. et al. Inferring steady state single-cell gene expression distributions from analysis of mesoscopic samples. Genome Biol 7 (2006).
Mary, P. et al. Analysis of gene expression at the single-cell level using microdroplet-based microfluidic technology. Biomicrofluidics 5 (2011).
Mazor, Y., Barnea, I., Keydar, I. & Benhar, I. Antibody internalization studied using a novel IgG binding toxin fusion. Journal of Immunological Methods 321, 41-59 (2007).
McDaniel, J. R., DeKosky, B. J., Tanno, H., Ellington, A. D. & Georgiou, G. Ultra-high-throughput sequencing of the immune receptor repertoire from millions of lymphocytes. Nat. Protoc. 11,429-442 (2016).
Mei, H.E. et al. Blood-borne human plasma cells in steady state are derived from mucosal immune responses. Blood 113, 2461-2469 (2009).
Meijer, P. et al. Isolation of human antibody repertoires with preservation of the natural heavy and light chain pairing. Journal of molecular biology 358, 764-772 (2006).
Mitchell, A. M. et al. Shared αβ Usage in Lungs of Sarcoidosis Patients with Löfgren's Syndrome. J. Immunol. 199,2279-2290 (2017).
Munson, D. J. et al. Identification of shared TCR sequences from T cells in human breast cancer using emulsion RT-PCR. Proc. Natl. Acad. Sci. U. S. A. 113,8272-7 (2016).
Nishioka, M. et al. Long and accurate PCR with a mixture of KOD DNA polymerase and its exonuclease deficient mutant enzyme. J. Biotechnol. 88, 141-149 (2001).
Novak, R. et al. Single-Cell Multiplex Gene Detection and Sequencing with Microfluidically Generated Agarose Emulsions. Angew. Chem.-Int. Edit. 50, 390-395 (2011).
Pinheiro, V. B. et al. Synthetic Genetic Polymers Capable of Heredity and Evolution. Science 336, 341-344 (2012).
Reddy, S. T. et al. Monoclonal antibodies isolated without screening by analyzing the variable-gene repertoire of plasma cells. Nature biotechnology 28, 965-U920 (2010).
Rajan et al. Recombinant human B cell repertoires enable screening for rare, specific, and natively paired antibodies. Communications Biology (2018).
Roberts, J. D., Bebenek, K. & Kunkel, T. A. The accuracy of reverse transcriptase from HIV-1. Science 242, 1171-1173 (1988).
Sanchez-Freire, V. et al. Microfluidic single-cell real-time PCR for comparative analysis of gene expression patterns. Nat. Protocols 7, 829-838 (2012).
Schmitt, M. W. et al. Detection of ultra-rare mutations by next-generation sequencing. Proc. Natl. Acad. Sci. 109, 14508-14513 (2012).
Smith, K. et al. Rapid generation of fully human monoclonal antibodies specific to a vaccinating antigen. Nat. Protocols 4, 372-384 (2009).
Takagi, M. et al. Characterization of DNA polymerase from Pyrococcus sp. strain KOD1 and its application to PCR. Appl. Environ. Microbiol. 63, 4504-4510 (1997).
Taubenheim, N. et al. High Rate of Antibody Secretion Is not Integral to Plasma Cell Differentiation as Revealed by XBP-1 Deficiency. The Journal of Immunology 189, 3328-3338 (2012).
Temin, H. M. & Mizutani, S. RNA-dependent DNA polymerase in virions of Rous sarcoma virus. Nature 226, 1211-1213 (1970).
Toriello, N.M. et al. Integrated microfluidic bioprocessor for single-cell gene expression analysis. Proc Natl Acad Sci USA 105, 20173-20178 (2008).
Trapnell, C. et al. Differential gene and transcript expression analysis of RNA-seq experiments with TopHat and Cufflinks. Nat. Protoc. 7,562-578 (2012).
Turchaninova, M. A. et al. Pairing of T-cell receptor chains via emulsion PCR. Eur. J. Immunol. 43,2507-2515 (2013).
Wang, A. H.-J. et al. Molecular structure of r(GCG)d(TATACGC): a DNA-RNA hybrid helix joined to double helical DNA. Nature 299, 601-604 (1982).
Wei, X. et al. Viral dynamics in human immunodeficiency virus type 1 infection. Nature 373, 117-122 (1995).
White, A.K. et al. High-throughput microfluidic single-cell RT-qPCR. Proc Natl Acad Sci U SA (2011).
Wrammert, J. et al. Rapid cloning of high-affinity human monoclonal antibodies against influenza virus. Nature 453, 667-671 (2008).
Wu, X. et al. Focused Evolution of HIV-1 Neutralizing Antibodies Revealed by Structures and Deep Sequencing. Science 333, 1593-1602 (2011).
Xiong, Y. & Eickbush, T. H. Origin and evolution of retroelements based upon their reverse transcriptase sequences. EMBO J 9, 3353-3362 (1990).

What is claimed is:

1. A method comprising:
a) sequestering single cells into individual compartments;
b) lysing the cells to generate a lysate comprising mRNA transcripts;
c) performing reverse transcription and a first PCR amplification of the mRNA transcripts using a single polymerase to generate distinct cDNA products corresponding to at least two distinct mRNAs from a single cell; and
d) sequencing the distinct cDNA products amplified from at least one single cell.

2. The method of claim 1, wherein the single polymerase has proofreading activity.

3. The method of claim 1, further defined as a method for obtaining a plurality of natively paired mRNA transcript sequences.

4. The method of claim 1, wherein the cells are B cells.

5. The method of claim 1, wherein the at least two distinct mRNAs encode paired antibody VH and VL sequences.

6. The method of claim 5, further defined as a method for obtaining paired antibody VH and VL sequences for an antibody that binds to an antigen of interest. The method of claim 1, wherein the cells are T cells.

8. The method of claim 1, wherein the at least two distinct mRNAs encode paired T-cell receptor sequences.

9. The method of claim 8, further defined as a method for obtaining paired T-cell receptor sequences for T-cell receptor that binds to an epitope of interest.

10. The method of claim 1, wherein the mRNA transcripts are not captured.

11. The method of claim 1, wherein the mRNA transcripts are bound to a solid support prior to step (c).

12. The method of claim 1, further comprising binding the mRNA transcripts to a solid support prior to step (c).

13. The method of claim 12, wherein the solid support is a bead.

14. The method of claim 12, wherein the solid support comprises oligonucleotides that hybridize to the mRNA transcripts.

15. The method of claim 12, wherein the oligonucleotides comprise poly-T sequences.

16. The method of claim 1, wherein the individual compartments are wells in a gel or microtiter plate.

17. The method of claim 1, said individual compartments having a volume of greater than 5 nL.

18. The method of claim 17, wherein the wells are sealed with a permeable membrane prior to step (c).

19. The method of claim 1, wherein the individual compartments are microvesicles in an emulsion.

20. The method of claim 1, wherein steps (a) and (b) are performed concurrently.

21. The method of claim 1, wherein steps (a) and (b) comprise isolating single cells into individual microvesicles in an emulsion and in the presence of a cell lysis solution.

22. The method of claim 1, wherein the individual compartments in step (a) further comprise oligonucleotides for priming of reverse transcription.

23. The method of claim 3, wherein step (b) further comprises allowing the mRNA transcripts to associate with the oligonucleotides.

24. The method of claim 3, comprising obtaining sequences from at least 10,000 individual cells.

25. The method of claim 4, comprising obtaining at least 5,000 individual paired antibody VH and VL sequences.

26. The method of claim 1, wherein step (c) comprises linking cDNA by performing overlap extension reverse transcriptase polymerase chain reaction to link at least two transcripts into a single DNA molecule.

27. The method of claim 1, wherein step (c) does not comprise the use of overlap extension reverse transcriptase polymerase chain reaction.

28. The method of claim 4, wherein step (c) comprises linking VH and VL cDNAs by performing overlap extension reverse transcriptase polymerase chain reaction to link VH and VL cDNAs in single molecules.

29. The method of claim 4, wherein step (c) does not comprise the use of overlap extension reverse transcriptase polymerase chain reaction and wherein the VH and VL cDNAs are separate molecules.

30. The method of claim 4, wherein the VH and VL sequences are obtained by sequencing of distinct molecules.

31. The method of claim 4, further comprising identifying the paired antibody VH and VL sequences comprises performing a probability analysis of the sequences.

32. The method of claim 31, wherein the probability analysis is based on the CDR-H3 or CDR-L3 sequences.

33. The method of claim 31, wherein identifying the paired antibody VH and VL sequences comprises comparing raw sequencing read counts.

34. The method of claim 1, wherein step (c) comprises linking cDNA by performing recombination.

35. The method of claim 1, further comprising performing a second PCR amplification after step (c) and before step (d).

36. The method of claim 1, wherein the cells are mammalian cells.

37. The method of claim 1, wherein the cells are selected from the group consisting of: B cells, T cells, NKT cells, and cancer cells.

38. The method of claim 1, wherein sequestering the single cells comprises introducing the cells to a device comprising a plurality of microwells so that the majority of cells are captured as single cells.

39. The method of claim 1, further comprising identifying multiple mRNA transcripts for a plurality of single cells based on the sequencing step (d).

40. The method of claim 3, further comprising isolating the mRNA transcripts prior to step (c).

41. The method of claim 3, further comprising determining natively paired transcripts using probability analysis.

42. The method of claim 41, wherein identifying the natively paired transcripts comprises comparing raw sequencing read counts.

43. The method of claim 1, wherein the single polymerase is a recombinant Archaeal Family-B polymerase that transcribes a template that is RNA and has one or more mutations compared to a wild-type Archaeal Family-B polymerase.

44. The method of claim 43, wherein the polymerase has one or more genetically engineered mutations compared to a wild-type Archaeal Family-B polymerase, the polymerase having an amino acid sequence at least 70%, 75%, 80%, 85%, 90%, 95%, 97%, 98%, or 99% identical to SEQ ID NO: 1 and in which one or more amino acid residues at a position selected from the group consisting of positions Y493, Y384, V389, 1521, E664 and G711 in the amino acid sequence shown in SEQ ID NO:1 or at a position corresponding to any of these positions, are substituted with another amino acid residue.

45. The method of claim 44, comprising an amino acid substitution corresponding to position Y493 to a leucine residue or a cysteine residue.

46. The method of claim 44, comprising an amino acid substitution corresponding to position Y493 to a leucine residue.

47. The method of claim 44, comprising an amino acid substitution corresponding to position Y384 to a phenylalanine residue, a leucine residue, an alanine residue, a cysteine residue, a serine residue, a histidine residue, an isoleucine residue, a methionine residue, an asparagine residue, or a glutamine residue.

48. The method of claim 47, comprising an amino acid substitution corresponding to position Y384 to a histidine residue or an isoleucine residue.

49. The method of claim 44, comprising an amino acid substitution corresponding to position V389 to a methionine residue, a phenylalanine residue, a threonine residue, a tyrosine residue, a glutamine residue, an asparagine residue, or a histidine residue.

50. The method of claim 44, comprising an amino acid substitution corresponding to position V389 to an isoleucine residue.

51. The method of claim 44, comprising an amino acid substitution corresponding to position 1521 to a leucine.

52. The method of claim 44, comprising an amino acid substitution corresponding to E664 is to a lysine residue.

53. The method of claim 44, comprising an amino acid substitution corresponding to position G711 to a leucine residue, a cysteine residue, a threonine residue, an arginine residue, a histidine residue, a glutamine residue, a lysine residue, or a methionine residue.

54. The method of claim 53, comprising an amino acid substitution corresponding to position G711 to a valine residue.

55. The method of any one of claims 44-54, in which an amino acid substitution at a position R97 in the amino acid sequence shown in SEQ ID NO:1 with another amino acid residue.

56. The method of any one of claims 44-55, in which one or more amino acid residues at a position selected from the group consisting of positions A490, F587, M137, K118, T514, R381, F38, K466, E734 and N735 in the amino acid sequence shown in SEQ ID NO:1 or at a position corresponding to any of these positions, are substituted with another amino acid residue.

57. The method of any one of claims 43-56, wherein the polymerase has proofreading activity.

58. The method of any one of claims 43-56, wherein the polymerase lacks proofreading activity.

59. The method of any one of claims 43-58, wherein the polymerase has thermophilic activity.

60. The method of any one of claims 43-58, wherein the polymerase transcribes at least 10 nucleotides from a RNA template.

61. The method of any one of claims 43-58, wherein the polymerase further transcribes a template that is 2′-OMethyl DNA.

62. The method of claim 43, wherein the polymerase has an amino acid sequence that is at least 70%, 75%, 80%, 85%, 90%, 95%, 97%, 98%, or 99% identical to the amino acid sequence of SEQ ID NO:1 and an amino acid substitution corresponding to an amino acid at positions 493, 384, 389, 97, 521, 711, 735, or a combination thereof

63. The method of claim 62, further comprising acid substitution corresponding to an amino acid at positions 664.

64. The method of claim 62, comprising an amino acid substitution corresponding to position 493 to a leucine residue, a cysteine residue, or a phenylalanine residue.

65. The method of claim 62, comprising an amino acid substitution corresponding to position 493 to a leucine residue.

66. The method of claim 62, comprising an amino acid substitution corresponding to position 493 to an isoleucine residue, a valine residue, an alanine residue, a histidine residue, a threonine residue, or a serine residue.

67. The method of claim 62, wherein the polymerase has an amino acid sequence that is at least 70%, 75%, 80%, 85%, 90%, 95%, 97%, 98%, or 99% identical to the amino acid sequence of SEQ ID NO:1 and an amino acid substitution corresponding to an amino acid at positions 493, 384, 389, 521, 711 or a combination thereof.

68. The method of claim 62, further comprising an amino acid substitution that corresponds to an amino acid at position 490, 587, 137, 118, 514, 381, 38, 466, 734, or a combination thereof.

69. The method of claim 62, comprising amino acid substitution corresponding to position 384 to a histidine residue or an isoleucine residue.

70. The method of claim 62, comprising an amino acid substitution corresponding to position 384 to a phenylalanine residue, a leucine residue, an alanine residue, a cysteine residue, a serine residue, a histidine residue, an isoleucine residue, a methionine residue, an asparagine residue, or a glutamine residue.

71. The method of claim 62, comprising an amino acid substitution corresponding to position 389 to an isoleucine residue or a leucine residue.

72. The method of claim 62, comprising an amino acid substitution corresponding to position 389 to a methionine residue, a phenylalanine residue, a threonine residue, a tyrosine residue, a glutamine residue, an asparagine residue, or a histidine residue.

73. The method of claim 63, wherein the amino acid substitution corresponding to position 664 is to a lysine residue or a glutamine residue.

74. The method of claim 62, comprising an amino acid substitution corresponding to position 97 to any amino acid residue other than arginine.

75. The method of claim 62, comprising an amino acid substitution corresponding to position 521 to a leucine.

76. The method of claim 62, comprising an amino acid substitution corresponding to position 521 to a phenylalanine residue, a valine residue, a methionine residue, or a threonine residue.

77. The method of claim 62, comprising an amino acid substitution corresponding to position 711 to a valine residue, a serine residue, or an arginine residue.

78. The method of claim 62, comprising an amino acid substitution corresponding to position 711 to a leucine residue, a cysteine residue, a threonine residue, an arginine residue, a histidine residue, a glutamine residue, a lysine residue, or a methionine residue.

79. The method of claim 62, comprising an amino acid substitution corresponding to position 735 to a lysine residue.

80. The method of claim 62, comprising an amino acid substitution corresponding to position 735 to an arginine residue, a glutamine residue, an arginine residue, a tyrosine residue, or a histidine residue.

81. The method of claim 68, wherein the amino acid substitution corresponding to position 490 is to a threonine residue.

82. The method of claim 68, wherein the amino acid substitution corresponding to position 490 is to a valine residue, a serine residue, or a cysteine residue.

83. The method of claim 68, wherein the amino acid substitution corresponding to position 587 is to a leucine residue or an isoleucine residue.

84. The method of claim 68, wherein the amino acid substitution corresponding to position 587 is to an alanine residue, a threonine residue, or a valine residue.

85. The method of claim 68, wherein the amino acid substitution corresponding to position 137 is to a leucine residue or an isoleucine residue.

86. The method of claim 68, wherein the amino acid substitution corresponding to position 137 is to an alanine residue, a threonine residue, or a valine residue.

87. The method of claim 68, wherein the amino acid substitution corresponding to position 118 is to an isoleucine residue.

88. The method of claim 68, wherein the amino acid substitution corresponding to position 118 is to a methionine residue, a valine residue, or a leucine residue.

89. The method of claim 68, wherein the amino acid substitution corresponding to position 514 is to an isoleucine residue.

90. The method of claim 68, wherein the amino acid substitution corresponding to position 514 is to a valine residue, a leucine residue, or a methionine residue.

91. The method of claim 68, wherein the amino acid substitution corresponding to position 381 is to a histidine residue.

92. The method of claim 68, wherein the amino acid substitution corresponding to position 381 is to a serine residue, a glutamine residue, or a lysine residue.

93. The method of claim 68, wherein the amino acid substitution corresponding to position 38 is to a leucine residue or an isoleucine residue.

94. The method of claim 68, wherein the amino acid substitution corresponding to position 38 is to a valine residue, a methionine residue, or a serine residue.

95. The method of claim 68, wherein the amino acid substitution corresponding to position 466 is to an arginine residue.

96. The method of claim 68, wherein the amino acid substitution corresponding to position 466 is to a glutamate residue, an aspartate residue, or a glutamine residue.

97. The method of claim 68, wherein the amino acid substitution corresponding to position 734 is to a lysine residue.

98. The method of claim 68, wherein the amino acid substitution corresponding to position 734 is to an arginine residue, a glutamine residue, or an asparagine residue.

99. The method of claim 43, wherein the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution at one or more of the following positions corresponding to SEQ ID NO:1: R97; Y384; V389; Y493; F587; E664; G711; and W768.

100. The method of claim 99, wherein the polymerase has one or more of the following amino acid substitutions corresponding to SEQ ID NO:1: R97M; Y384H; V3891; Y493L; F587L; E664K; G711V; and W768R.

101. The method of claim 43, wherein the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution at one or more of the following positions corresponding to SEQ ID NO:1: F38; R97; K118; R381; Y384; V389; Y493; T514; F587; E664; G711; and W768.

102. The method of claim 101, wherein the polymerase has one or more of the following amino acid substitutions corresponding to SEQ ID NO:1: F38L; R97M; K1181; R381H; Y384H; V389I; Y493L; T514I; F587L; E664K; G711V; and W768R.

103. The method of claim 43, wherein the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution at one or more of the following positions corresponding to SEQ ID NO:1: F38; R97; K118; M137; R381; Y384; V389; K466; Y493; T514; F587; E664; G711; and W768.

104. The method of claim 103, wherein the polymerase has one or more of the following amino acid substitutions corresponding to SEQ ID NO:1: F38L; R97M; K1181; M137L; R381H; Y384H; V389I; K466R; Y493L; T514I; F587L; E664K; G711V; and W768R.

105. The method of claim 43, wherein the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution at one or more of the following positions corresponding to SEQ ID NO:1: F38; R97; K118; M137; R381; Y384; V389; K466; Y493; T514; 1521; F587; E664; G711; N735; and W768.

106. The method of claim 105, wherein the polymerase has one or more of the following amino acid substitutions corresponding to SEQ ID NO:1: F38L; R97M; K1181; M137L; R381H; Y384H; V389I; K466R; Y493L; T514I; I521L; F587L; E664K; G711V; N735K; and W768R.

107. The method of any one of claims 43-106, wherein the polymerase further comprises an additional domain.

108. The method of claim 107, wherein the additional domain has polymerization enhancing activity.

109. The method of claim 107, wherein the additional domain comprise part or all of DNA-binding protein 7d (Sso7d), Proliferating cell nuclear antigen (PCNA), helicase, single stranded binding proteins, bovine serum albumin (BSA), one or more affinity tags, one or more labels, and a combination thereof.

110. The method of any one of claims 43-106, wherein the polymerase lacks 3′ to 5′ exonuclease activity.

111. The method of claim 110, wherein the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution corresponding to N210.

112. The method of claim 111, wherein the polymerase has an amino acid substitution corresponding to N210D.

113. The method of claim 110, wherein the polymerase has an amino acid sequence that is at least 70%, 75%, 80%, 85%, 90%, 95%, 97%, 98%, or 99% identical to the amino acid sequence of SEQ ID NO:1 and wherein the polymerase has an amino acid substitution corresponding to D141 and E143.

114. The method of claim 113, wherein the polymerase has an amino acid substitution corresponding to D141A and E143A.

115. The method of claim 43, wherein the polymerase comprises an amino acid sequence 98% identical to the amino acid sequence of SEQ ID NO: 3.

116. The method of claim 115, wherein the polymerase comprises an amino acid sequence 99% identical to the amino acid sequence of SED ID NO: 3.

117. The method of claim 116, wherein the polymerase comprises an amino acid sequence identical to the amino acid sequence of SEQ ID NO: 3.

118. A composition isolated in a compartment comprising:
(i) polymerase that comprises one or more genetically engineered mutations compared to a wild-type Archaeal Family-B polymerase, the polymerase having an amino acid sequence at least 70%, 75%, 80%, 85%, 90%, 95%, 97%, 98%, or 99% identical to SEQ ID NO: 1 and in which one or more amino acid residues at a position selected from the group consisting of positions Y493, Y384, V389, 1521, E664 and G711 in the amino acid sequence shown in SEQ ID NO:1 or at a position corresponding to any of these positions, are substituted with another amino acid residue; and
(ii) a DNA molecule comprising linked cDNAs corresponding to two distinct mRNA transcripts from a single cell.

119. The composition of claim 118, wherein the compartment is an emulsion macrovesicle.

120. The composition of claim 118, wherein the two distinct mRNA transcripts encode paired antibody VH and VL domains.

121. The composition of claim 118, wherein the two distinct mRNA transcripts encode paired T-cell receptor sequences.
